(19) United States
(12) Patent Application Publication
(10) Pub. No.: US 2024/0247285 A1
(43) Pub. Date: Jul. 25, 2024

(54) METHODS FOR DELIVERING GENOME EDITING MOLECULES TO THE NUCLEUS OR CYTOSOL OF A CELL AND USES THEREOF

(71) Applicant: SQZ Biotechnologies Company, Watertown, MA (US)

(72) Inventors: Jacquelyn L. SIKORA HANSON, Watertown, MA (US); Marija TADIN-STRAPPS, Watertown, MA (US)

(73) Assignee: SQZ Biotechnologies Company, Watertown, MA (US)

(21) Appl. No.: 18/559,925

(22) PCT Filed: May 10, 2022
(86) PCT No.: PCT/US2022/028546

§ 371 Date: Nov. 9, 2023

Related U.S. Application Data
(60) Provisional application No. 63/186,651, filed on May 10, 2021.

(51) Int. Cl. C12N 15/87; A61K 35/17; C12N 5/0783; C12N 9/22; C12N 15/11

(52) U.S. Cl. CPC C12N 15/87; A61K 35/17; C12N 5/0636; C12N 9/22; C12N 15/11; C12N 2310/20

(57) ABSTRACT

The present disclosure provides methods for delivering one or more payloads (e.g., gene-editing pay load) to a cell, wherein the method comprises passing a cell suspension comprising the cell and the pay load through one or more constrictions, wherein the one or more constrictions deform the cell, thereby causing a perturbation of the cell such that the one or more pay loads enters the cell.

### CROSS-REFERENCE TO RELATED APPLICATION

This PCT application claims the priority benefit of U.S. Provisional Application No. 63/186,651, filed on May 10, 2021, which is herein incorporated by reference in its entirety.

### FIELD OF DISCLOSURE

The present disclosure relates generally to methods of delivering one or more payloads (e.g., capable of modulating the expression of one or more genes) to a cell (e.g., to the nucleus of the cell) through the use of one or more constrictions.

### BACKGROUND OF DISCLOSURE

Intracellular delivery to specific compartments of a cell (e.g., nucleus) is central to the success of modern medicine, such as gene therapy and genetic engineering. Existing technologies aimed at intracellular delivery rely on electrical fields, nanoparticles, and/or pore-forming chemicals. However, such methods suffer from numerous complications, including non-specific molecule delivery, modification or damage to the payload molecules, high cell death, low throughput, and/or difficult implementation. Due to their large size, complexes composed of biomolecules such as polypeptides, nucleic acids, carbohydrates, lipids, and/or small molecules cannot readily cross the cellular membrane. Thus, delivery of such complexes has been a challenge and there remains a need for improved techniques that are much more effective at delivering complexes to a variety of cell types.

### SUMMARY OF DISCLOSURE

Provided herein is a method of delivering a payload to the nucleus of a cell, comprising passing a cell suspension comprising (i) the cell and (ii) the payload through a plurality of constrictions under one or more parameters, wherein passing the cell suspension through the plurality of constrictions allows the payload to enter the cell and be delivered to the nucleus.

In some aspects, the payload comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

In some aspects, the payload comprises a protein-nucleic acid complex. In some aspects, the payload comprises a gene editing tool. In some aspects, the gene editing tool comprises a shRNA, siRNA, miRNA, antisense oligonucleotides, zinc-finger nuclease, meganuclease, transcription activator-like effector nuclease (TALEN), a CRISPR/Cas system, a ribonucleoprotein (RNP), a Cre recombinase, a lipid nanoparticle or any combination thereof. In some aspects, the gene editing tool is a CRISPR/Cas system. In some aspects, the CRISPR/Cas system comprises a Cas9 nuclease. In some aspects, the protein-nucleic complex comprises a ribonucleotide protein and a mRNA. In some aspects, the gene editing tool is capable of modulating the expression of a gene selected from a beta-2 microglobulin (B2M), a T-cell immunoglobulin and mucin-domain containing-3 (TIM3), a T-cell receptor alpha constant (TRAC), CD86, TGF-β, PD-1, BC11a, CCR5, CD38, CISH, or combinations thereof.

In some aspects, the delivery of the payload to the nucleus occurs less than about 1 hour, less than about 50 minutes, less than about 40 minutes, less than about 30 minutes, less than about 20 minutes, less than about 10 minutes, less than about 5 minutes, less than about 4 minutes, less than about 3 minutes, less than about 2 minutes, less than about 1 minute, less than about 30 seconds, less than about 20 seconds, less than about 10 seconds, less than about 5 seconds, or less than about 1 second after the payload enters the cell.

Also provided herein is a method of increasing the delivery efficiency of a payload to the nucleus of a cell, comprising passing a cell suspension comprising (i) the cell and (ii) the payload through a plurality of constrictions under one or more parameters, wherein passing the cell suspension through the plurality of constrictions allows greater delivery of the payload to the nucleus of the cell.

In some aspects, the delivery efficiency of the payload to the nucleus of the cell is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, compared to a reference delivery efficiency. In some aspects, the reference delivery efficiency comprises: (i) the delivery efficiency of the payload to the nucleus of the cell after passing the cell through a single constriction; (ii) the delivery efficiency of the payload when delivered to the nucleus using a method that does not comprising passing the cell through a constriction; or (iii) both (i) and (ii).

In any of the above methods directed to increasing the delivery efficiency of a payload to the nucleus of a cell, in some aspects, the payload comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

In some aspects, the payload comprises a protein-nucleic acid complex. In some aspects, the payload comprises a gene editing tool. In some aspects, the gene editing tool comprises a shRNA, siRNA, miRNA, antisense oligonucleotides, zinc-finger nuclease, meganuclease, transcription activator-like effector nuclease (TALEN), a CRISPR/Cas system, a ribonucleoprotein (RNP), a Cre recombinase, a lipid nanoparticle, or any combination thereof. In some aspects, the gene editing tool is a CRISPR/Cas system. In some aspects, CRISPR/Cas system comprises a Cas9 nuclease. In some aspects, the protein-nucleic complex comprises a ribonucleotide protein and a mRNA. In some aspects, the gene editing tool is capable of modulating the expression of a gene selected from a beta-2 microglobulin (B2M), a T-cell immunoglobulin and mucin-domain containing-3 (TIM3), a T-cell receptor alpha constant (TRAC), or combinations thereof.

In any of the methods provided above, in some aspects, the plurality of constrictions are contained within a single microfluidic chip. In some aspects, wherein the plurality of constrictions are contained within multiple microfluidic chips, wherein each of the multiple microfluidic chips comprises a constriction. In some aspects, each of the multiple microfluidic chips are the same. In some aspects, one or more of the multiple microfluidic chips are different.

In any of the methods provided above, in some aspects, the time interval between passing the cell suspension through a first constriction and a second constriction of the plurality of constrictions is less than about 1 μs, less than about 1 second, less than about 1 minute, less than about 30 minutes, less than about 1 hour, less than about 6 hours, less than about 12 hours, less than about 1 day, less than about 2 days, less than about 3 days, less than about 4 days, or less than about 5 days.

In some aspects, the plurality of constrictions comprises at least about 2, at least about 3, at least about 4, at least about 5, at least about 6, at least about 7, at least about 8, at least about 9, at least about 10, at least about 20, at least about 30, at least about 40, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1,000 or more separate constrictions. In some aspects, each of the plurality of constrictions is the same. In some aspects, one or more of the plurality of constrictions are different. In some aspects, one or more of the plurality of constrictions differ in their length, depth, width, or combinations thereof.

The present disclosure further provides a method of concurrently delivering multiple payloads to a cell, comprising passing a cell suspension comprising (i) the cell and (ii) the multiple payloads through a constriction under one or more parameters, wherein passing the cell suspension through the constriction allows the multiple payloads to enter the cells.

In some aspects, the multiple payloads comprise about 2, about 3, about 4, about 5, about 6, about 7, about 8, about 9, or about 10 types of payloads. In some aspects, each of the multiple payloads is different. In some aspects, the multiple payloads comprise a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

In some aspects, the multiple payloads comprise a protein-nucleic acid complex. In some aspects, the payload comprises a gene editing tool. In some aspects, the gene editing tool comprises a shRNA, siRNA, miRNA, antisense oligonucleotides, zinc-finger nuclease, meganuclease, transcription activator-like effector nuclease (TALEN), a CRISPR/Cas system, a ribonucleoprotein (RNP), a Cre recombinase, a lipid nanoparticle, or any combination thereof. In some aspects, the gene editing tool is a CRISPR/Cas system. In some aspects, CRISPR/Cas system comprises a Cas9 nuclease. In some aspects, the protein-nucleic complex comprises a ribonucleotide protein and a mRNA. In some aspects, the gene editing tool is capable of modulating the expression of a gene selected from a beta-2 microglobulin (B2M), a T-cell immunoglobulin and mucin-domain containing-3 (TIM3), a T-cell receptor alpha constant (TRAC), or combinations thereof.

Also provided herein is a method of sequentially delivering a first payload and a second payload to a cell, comprising passing a cell suspension comprising (i) the cell and (ii) the first and second payloads through a first constriction and a second constriction under one or more parameters, wherein passing the cell suspension through the first constriction allows the first payload to enter the cell, and wherein passing the cell suspension through the second constriction allows the second payload to enter the cell.

In some aspects, the cell suspension is passed through the second constriction at least about 1 minute, at least about 30 minutes, at least about 1 hour, at least about 6 hours, at least about 12 hours, at least about 1 day, at least about 2 days, or at least about 3 days after the cell suspension is passed through the first constriction.

In some aspects, the first payload, the second payload, or both the first and second payloads comprise a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

In some aspects, the first payload, the second payload, or both the first and second payloads comprise a protein-nucleic acid complex. In some aspects, the first payload, the second payload, or both the first and second payloads comprise a gene editing tool. In some aspects, the gene editing tool comprises a shRNA, siRNA, miRNA, antisense oligonucleotides, zinc-finger nuclease, meganuclease, transcription activator-like effector nuclease (TALEN), a CRISPR/Cas system, a ribonucleoprotein (RNP), a Cre recombinase, a lipid nanoparticle, or any combination thereof. In some aspects, the gene editing tool is a CRISPR/Cas system. In some aspects, CRISPR/Cas system comprises a Cas9 nuclease. In some aspects, the protein-nucleic complex comprises a ribonucleotide protein and a mRNA. In some aspects, the gene editing tool is capable of modulating the expression of a gene selected from a beta-2 microglobulin (B2M), a T-cell immunoglobulin and mucin-domain containing-3 (TIM3), a T-cell receptor alpha constant (TRAC), or combinations thereof.

In some aspects, the first payload and the second payload are different. In some aspects, the first payload and the second payload are the same. In some aspects, the first constriction and the second constriction are different. In some aspects, the first constriction and the second constriction are the same. In some aspects, the first constriction and the second constriction are contained with a single microfluidic chip. In some aspects, the first constriction and the second constriction are contained within separate microfluidic chips.

In some aspects, the cell is not in contact with the second payload when the cell suspension is passed through the first constriction. In some aspects, the cell is not in contact with the first payload when the cell suspension is passed through the second constriction.

Provided herein is a method of modulating the expression of a gene in a cell, comprising passing a cell suspension comprising (i) the cell and (ii) a payload through a plurality of constrictions under one or more parameters, wherein passing the cell suspension through the plurality of constrictions allows the payload to enter the cell and be delivered to the nucleus of the cell, and wherein the payload is capable of modulating the expression of the gene.

In the above method, in some aspects, the payload comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

In some aspects, the payload comprises a protein-nucleic acid complex. In some aspects, the payload comprises a gene editing tool. In some aspects, the gene editing tool comprises a shRNA, siRNA, miRNA, antisense oligonucleotides, zinc-finger nuclease, meganuclease, transcription activator-like effector nuclease (TALEN), a CRISPR/Cas system, a ribonucleoprotein (RNP), a Cre recombinase, a lipid nanoparticle, or any combination thereof. In some aspects, the gene editing tool is a CRISPR/Cas system. In some aspects, CRISPR/Cas system comprises a Cas9 nuclease. In some aspects, the protein-nucleic complex comprises a ribonucleotide protein and a mRNA. In some aspects, the gene editing tool is capable of modulating the expression of a gene selected from a beta-2 microglobulin (B2M), a T-cell immunoglobulin and mucin-domain containing-3 (TIM3), a T-cell receptor alpha constant (TRAC), or combinations thereof.

In the above methods directed to modulating the expression of a gene in a cell, in some aspects, the expression of the gene in the cell is reduced by at least about 5%, at least about 10%, at least about 20%, at least about 30%, at least about 40%, at least about 50%, at least about 60%, at least about 70%, at least about 80%, at least about 90% or more after the delivery of the payload to the nucleus of the cell. In some aspects, the expression of the gene in the cell is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold or more after the delivery of the payload to the nucleus of the cell.

In some aspects, the time interval between passing the cell suspension through a first constriction and a second constriction of the plurality of constrictions is at least about 1 minute, at least about 30 minutes, at least about 1 hour, at least about 6 hours, at least about 12 hours, at least about 1 day, at least about 2 days, or at least about 3 days.

In some aspects, the plurality of constrictions comprises at least about 2, at least about 3, at least about 4, at least about 5, at least about 6, at least about 7, at least about 8, at least about 9, at least about 10, at least about 20, at least about 30, at least about 40, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1,000 or more separate constrictions. In some aspects, each of the plurality of constrictions is the same. In some aspects, one or more of the plurality of constrictions are different. In some aspects, the one or more of the plurality of constrictions differ in their length, depth, width, or combinations thereof.

In any of the above methods, in some aspects, the cell comprises a stem cell, a somatic cell, or both. In some aspects, the stem cell comprises an induced pluripotent stem cell (iPSC), an embryonic stem cell, a tissue-specific stem cell, a mesenchymal stem cell, or combinations thereof. In some aspects, the somatic cell comprises a blood cell. In some aspects, the blood cell comprises PBMC. In some aspects, the PBMC comprises an immune cell. In some aspects, the immune cell comprises a T cell, a B cell, a natural killer (NK) cell, a dendritic cell (DC), a NKT cell, a mast cell, a monocyte, a macrophage, a basophil, an eosinophil, a neutrophil, a DC2.4 dendritic cell, or combinations thereof.

In any of the above methods, in some aspects, the one or more parameters are selected from a cell density; pressure; length, width, and/or depth of the constriction; diameter of the constriction; diameter of the cells; temperature; entrance angle of the constriction; exit angle of the constriction; length, width, and/or width of an approach region; surface property of the constriction (e.g., roughness, chemical modification, hydrophilic, hydrophobic); operating flow speed; payload concentration; viscosity, osmolarity, salt concentration, serum content, and/or pH of the cell suspension; time in the constriction; shear rate in the constriction; type of payload, or combinations thereof.

In some aspects, the cell density is at least about 1×10³ cells/mL, at least about 1×10⁴ cells/mL, at least about 1×10⁵ cells/mL, at least about 1×10⁶ cells/mL, at least about 2×10⁶ cells/mL, at least about 3×10⁶ cells/mL, at least about 4×10⁶ cells/mL, at least about 5×10⁶ cells/mL, at least about 6×10⁶ cells/mL, at least about 7×10⁶ cells/mL, at least about 8×10⁶ cells/mL, at least about 9×10⁶ cells/mL, at least about 6×10⁷ cells/mL, at least about 7×10⁷ cells/mL, at least about 8×10⁷ cells/mL, at least about 9×10⁷ cells/mL, at least about 1×10⁸ cells/mL, at least about 1.1×10⁸ cells/mL, at least about 1.2×10⁸ cells/mL, at least about 1.3×10⁸ cells/mL, at least about 1.4×10⁸ cells/mL, at least about 1.5×10⁸ cells/mL, at least about 2.0×10⁸ cells/mL, at least about 3.0×10⁸ cells/mL, at least about 4.0×10⁸ cells/mL, at least about 5.0×10⁸ cells/mL, at least about 6.0×10⁸ cells/mL, at least about 7.0×10⁸ cells/mL, at least about 8.0×10⁸ cells/mL, at least about 9.0×10⁸ cells/mL, or at least about 1.0×10⁹ cells/mL or more.

In some aspects, the pressure is at least about 1 psi, at least about 2 psi, at least about 3 psi, at least about 4 psi, at least about 5 psi, at least about 6 psi, at least about 7 psi, at least about 8 psi, at least about 9 psi, at least about 10 psi, at least about 20 psi, at least about 30 psi, at least about 35 psi, at least about 40 psi, at least about 45 psi, at least about 50 psi, at least about 55 psi, at least about 60 psi, at least about 65 psi, at least about 70 psi, at least about 75 psi, at least about 80 psi, at least about 85 psi, at least about 90 psi, at least about 95 psi, at least about 100 psi, at least about 110 psi, at least about 120 psi, at least about 130 psi, at least about 140 psi, or at least about 150 psi.

In some aspects, the diameter of the constriction is about 20%, about 30%, about 40%, about 50%, about 60%, about 70%, about 80%, about 90%, or about 99% of the diameter of the cell.

In some aspects, the length of the constriction is up to 100 μm. In some aspects, the length of the constriction is less than about 1 μm, less than about 5 μm, less than about 10 μm, less than about 20 μm, less than about 30 μm, less than about 40 μm, less than about 50 μm, less than about 60 μm, less than about 70 μm, less than about 80 μm, less than about 90 μm, or less than about 100 μm. In some aspects, the length of the constriction is about 1 μm, about 5 μm, about 10 μm, about 20 μm, about 30 μm, about 40 μm, about 50 μm, about 60 μm, about 70 μm, about 80 μm, about 90 μm, or about 100 μm.

In some aspects, the width of the constriction is up to about 10 μm. In some aspects, the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm. In some aspects, the width of the constriction is between about 2 μm to about 10 μm. In some aspects, the width of the constriction is about 2 μm, about 3 μm, about 4 μm, about 5 μm, about 6 μm, about 7 μm, about 8 μm, about 9 μm, or about 10 μm.

In some aspects, the depth of the constriction is at least about 1 μm. In some aspects, the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm. In some aspects, the depth of the constriction is about 5 μm to about 90 μm. In some aspects, the depth is about 5 μm, about 10 μm, about 20 μm, about 30 μm, about 40 μm, about 50 μm, about 60 μm, about 70 μm, about 80 μm, or about 90 μm.

In some aspects, the length of the constriction is about 30 μm, the width of the constriction is about 4 μm, and the depth of the constriction is about 70 μm.

Also provided herein is a cell comprising one or more payloads, wherein the one or more payloads were delivered to the cell using any of the methods provided herein.

Provided herein is a composition comprising the cell described above, and a pharmaceutically acceptable carrier. Provided herein is a kit comprising the cell described above, and instructions for use.

Present disclosure provides a method of treating a disease or disorder in a subject in need thereof, comprising administering to the subject any of the cell or composition described herein.

Provided herein is a composition comprising a population of cells, which have been modified to comprise one or more payloads, wherein the one or more payloads were delivered to the population of cells using any of the methods provided herein.

### BRIEF DESCRIPTION OF FIGURES

FIGS. 1A and 1B show surface expression of B2M and CD86 expression in T cells after delivery of one of the following payloads using a single squeeze processing: (i) B2M-specific RNPs alone; (ii) CD86 mRNA alone; and (iii) combination of B2M-specific RNPs and CD86 mRNA (“multiplexed”). In the combination group, the B2M-specific RNPs and the CD86 mRNA were co-delivered (i.e., concurrently). Cells that that were passed through the constriction but without any payload were used as control (“squeeze alone”). FIG. 1A provides the results as stacked bar graphs, and shows the percentage of total T cells having the following phenotypes from the different groups: (i) expressing B2M alone (“B2M+CD86−”), (ii) expressing neither B2M nor CD86 (“B2M-CD86−”), (iii) expressing both B2M and CD86 (“B2M+CD86+”), and (iv) expressing CD86 alone (“B2M-CD86+”). FIG. 1B provides flow cytometry plots of the same results.

FIGS. 2A-2C show surface expression of B2M, TIM-3, and TRAC, respectively, in T cells after delivery of one of the following payloads using a single squeeze processing: (i) non-targeting RNP alone; (ii) B2M-specific RNPs; (iii) TRAC-specific RNPs; (iv) TIM-3-specific RNPs; and (v) combination of B2M-specific RNPs, TRAC-specific RNPs, and TIM-3-specific RNPs. For the combination group, the different RNPs were co-delivered to the cells. Normal cells (i.e., cells that did not undergo squeeze processing) (“NC”) and cells that underwent squeeze processing but without any RNPs (“squeeze alone”) were used as controls. The expression of B2M, TIM-3, and TRAC are shown as a percentage of total T cells at three days after the last squeeze processing.

FIG. 2D shows the surface knock-down efficiency of B2M, TIM-3, and/or TRAC expression in T cells after co-delivery of B2M-specific RNPs, TIM-3-specific RNPs, and TRAC-specific RNPs using a single squeeze processing. Cells that underwent squeeze processing but without any RNPs were used as control (“squeeze alone”). “No-Negative” corresponds to T cells that expressed all three proteins (i.e., B2M, TIM-3, and TRAC). “Single-Negative” corresponds to T cells that expressed only two of the three proteins. “Double-Negative” corresponds to T cells that expressed only one of the three proteins. “Triple-Negative” corresponds to T cells that did not express any of the three proteins.

FIGS. 3A-3C show surface expression of CD3 (proxy for TRAC expression), TIM-3, and B2M, respectively, after sequential delivery of the following payloads using squeeze processing: (i) TRAC-specific RNPs, (ii) TIM-3-specific RNPs, and (iii) B2M-specific RNPs. CD3 and B2M expression are shown as a percentage of total T cells at three days after the last (i.e., third squeeze processing). TIM-3 expression is shown relative to the corresponding expression on non-squeezed cells (i.e., cells that did not undergo squeeze processing). In all, the T cells underwent three separate squeeze processings (i.e., 1^{st} squeeze, 2^{nd} squeeze, and 3^{rd} squeeze). For each of the three squeeze processings, the left bar represents cells that underwent squeeze processing without any payload (i.e., squeeze alone); the middle bar represents cells that underwent squeeze processing but with a non-targeting RNP; and the right bar represents cells that underwent squeeze processing with one of the targeted RNPs. Normal cells (i.e., cells that did not undergo squeeze processing) were used as control (1^{st} bar in each of FIGS. 3A, 3B, and 3C).

FIG. 4A shows the surface knock-down efficiency of B2M, TIM-3, and TRAC expression in T cells after delivery of TRAC-specific RNPs, TIM-3-specific RNPs, and B2M-specific RNPs using sequential squeeze processing. Cells that underwent squeeze processing but without any RNPs were used as control (“squeeze alone”). “No-Negative” corresponds to T cells that expressed all three proteins (i.e., B2M, TIM-3, and TRAC). “Single-Negative” corresponds to T cells that expressed only two of the three proteins. “Double-Negative” corresponds to T cells that expressed only one of the three proteins. “Triple-Negative” corresponds to T cells that did not express any of the three proteins.

FIG. 4B shows the percentage of total T cells having one of the following phenotype after delivery of TRAC-specific RNPs, TIM-3-specific RNPs, and B2M-specific RNPs using sequential squeeze processing: (i) expressing both B2M and TRAC (“B2M+TRAC+), (ii) expressing TRAC alone (“B2M-TRAC+”), (iii) expressing B2M alone (“B2M+TRAC−”), and (iv) expressing neither B2M nor TRAC (“B2M-TRAC−”). In all, the T cells underwent three separate squeeze processings (i.e., 1^{st} squeeze (2^{nd} to 4^{th} bars), 2^{nd} squeeze (5^{th} to 7^{th} bars), and 3^{rd} squeeze (8^{th} to 10^{th} bars)). For each of the three squeeze processings, the left bar represents cells that underwent squeeze processing only (i.e., no RNPs) (“squeeze alone”); the middle bar represents cells that underwent squeeze processing but with a non-targeting RNP (“NT RNP”); and the right bar represents cells that underwent squeeze processing with one of the targeted RNPs. Normal cells (i.e., cells that did not undergo squeeze processing) were used as control (1^{st} bar; “untreated”).

FIG. 5A shows a comparison of TRAC surface expression in T cells (i) after delivery of TRAC-specific RNP alone using squeeze processing (“TRAC RNP”) or (ii) after co-delivery of TRAC-specific RNP in combination with other RNPs (i.e., TIM-3-specific RNPs and B2M-specific RNPs) using squeeze processing (“Multiplexed”). As controls, normal cells (i.e., did not undergo squeeze processing) (“untreated”), cells that underwent squeeze processing but without any RNPs (“squeeze alone”), and cells that underwent squeeze processing but with non-targeting RNPs (“Non-targeting RNP”) were used as controls.

FIG. 5B shows the effect of sequential squeeze processing on cell viability. The number of live T cells, as quantified with flow cytometry based on Live/Dead staining, are provided after the 1^{st} squeeze processing (“1^{st} squeeze”), the 2^{nd} squeeze processing (“2^{nd} squeeze”), and the 3^{rd} squeeze processing (“3^{rd} squeeze”). For each of the squeeze processings shown, the bar to the right represents cells that received one of the targeted RNPs (i.e., TRAC-specific RNP, TIM-3-specific RNPs, and/or B2M-specific RNPs), and the bar to the left represents cells that underwent squeeze processing but without any RNPs (i.e., squeeze alone).

FIGS. 6A-6D show the editing of B2M in T cells after delivery one of the following payloads using squeeze processing: (i) no payload (i.e., underwent squeeze processing without any RNPs) (“squeeze alone”); (ii) B2M-specific RNP alone; (iii) TIM-3-specific RNP alone; (iv) TRAC-specific RNP alone; and (v) combination of B2M-specific RNP, TIM-3-specific RNP, and TRAC-specific RNP. For the combination group, the different RNPs were either co-delivered to the cells using a single squeeze processing (“multiplex RNP”) or delivered to the cells using sequential squeeze processing (“sequential RNP”). Normal cells (i.e., cells that did not undergo squeeze processing) were used as control (“no contact”). In FIG. 6A, B2M surface expression is shown as % of total T cells, measured using flow cytometry. In FIGS. 6B, 6C, and 6D, B2M gene editing is shown based on PCR sequence analysis submitted to TIDE and ICE.

FIGS. 7A-7E show the editing of TIM-3 in T cells after delivery one of the following payloads using squeeze processing: (i) no payload (i.e., underwent squeeze processing without any RNPs) (“squeeze alone”); (ii) B2M-specific RNP alone; (iii) TIM-3-specific RNP alone; (iv) TRAC-specific RNP alone; and (v) combination of B2M-specific RNP, TIM-3-specific RNP, and TRAC-specific RNP. For the combination group, the different RNPs were either co-delivered to the cells using a single squeeze processing (“multiplex RNP”) or delivered to the cells using sequential squeeze processing (“sequential RNP”). Normal cells (i.e., cells that did not undergo squeeze processing) were used as control (“no contact”). In FIG. 7A, B2M surface expression is shown as % of total T cells, measured using flow cytometry. In FIGS. 7B, 7C, 7D, and 7E, B2M gene editing is shown based on PCR sequence analysis submitted to TIDE and ICE. For each of FIG. 7B-7E, 1st bar corresponds to single RNP, 2^{nd} bar corresponds to multiple RNP, and the 3rd bar corresponds to sequential RNP.

FIGS. 8A-8D show the editing of CD3 as a TRAC proxy in T cells after delivery one of the following payloads using squeeze processing: (i) no payload (i.e., underwent squeeze processing without any RNPs) (“squeeze alone”); (ii) B2M-specific RNP alone; (iii) TIM-3-specific RNP alone; (iv) TRAC-specific RNP alone; and (v) combination of B2M-specific RNP, TIM-3-specific RNP, and TRAC-specific RNP. For the combination group, the different RNPs were either co-delivered to the cells using a single squeeze processing (“multiplex RNP”) or delivered to the cells using sequential squeeze processing (“sequential RNP”). Normal cells (i.e., cells that did not undergo squeeze processing) were used as control. In FIG. 8A, CD3 surface expression is shown as percentage of total T cells, measured by flow cytometry. In FIGS. 8B, 8C, and 8D, TRAC gene editing is shown based on PCR sequence analysis submitted to TIDE and ICE. For each of FIGS. 8B-8D, 1^{st} bar corresponds to single RNP, 2^{nd} bar corresponds to multiple RNP, and the 3^{rd} bar corresponds to sequential RNP.

FIG. 9 shows the surface knock-down efficiency of B2M, TIM-3, and/or TRAC expression in T cells after delivery of the B2M-specific RNP, TIM-3-specific RNP, and TRAC-specific RNP using sequential squeeze processing (“sequential RNP”). For comparison, surface knock-down efficiency for the following T cells are also provided: (i) T cells after co-delivery of the three RNPs (i.e., in combination using a single squeeze processing) (“multiplex RNP”); (ii) T cells that underwent squeeze processing alone without any RNPs (“squeeze alone”); and (iii) T cells that did not undergo squeeze processing (“untreated”). “No-Negative” corresponds to T cells that expressed all three proteins (i.e., B2M, TIM-3, and TRAC). “Single-Negative” corresponds to T cells that expressed only two of the three proteins. “Double-Negative” corresponds to T cells that expressed only one of the three proteins. “Triple-Negative” corresponds to T cells that did not express any of the three proteins.

FIGS. 10A and 10B provide comparison of B2M, TRAC, and TIM-3 gene knock out efficiency, as measured using 10× genomics deep sequencing analysis, in T cells after sequential (FIG. 10A) or multiplex (FIG. 10B) delivery of the three RNPs (i.e., specific for B2M, TIM-3, or TRAC) using squeeze processing.

FIG. 11 shows the frequency of T cells with expression below the threshold for each of the target genes B2M, TIM-3 and TRAC. The threshold was the theoretical baseline gene expression count based on untreated cells that underwent squeeze processing alone without any RNPs (i.e., squeeze alone; “control”) and sorted for partial transcripts. The B2M-specific RNP, TIM-3-specific RNP, and TRAC-specific RNP were either delivered to the cells concurrently using a single squeeze processing (i.e., co-delivery; “multiplex”) or delivered separately using sequential squeeze processing (“sequential”).

### DETAILED DESCRIPTION OF DISCLOSURE

The present disclosure is generally directed to methods of delivering one or more payloads (e.g., gene-editing payloads) to a cell. More particularly, in some aspects, the delivery methods provided herein comprise passing a cell suspension through a plurality of constrictions under one or more parameters, wherein passing the cell suspension through the plurality of constrictions allows a payload to enter the cell. In some aspects, the delivery method comprises a single squeeze processing, e.g., the plurality of constrictions are contained within a single chip. In some aspects, the delivery method comprises multiple squeeze processings, e.g., using multiple chips which comprise one or more constrictions. As demonstrated herein, such methods are effective in delivering one or more payloads to specific compartments of a cell (e.g., the nucleus), which can be particularly useful in therapy, e.g., when seeking to genetically modify the genome of a cell (e.g., for gene therapy). Additionally, the delivery methods provided herein can also be used to target multiple types of payloads (e.g., proteins and nucleic acids) into a cell. The multiple payloads can be delivered to the cells sequentially or concurrently. As described further else wherein the present disclosure, the delivery methods described herein (e.g., sequential delivery) are much more efficacious and exhibit less adverse effects (e.g., by reducing potential for risk of multiple simultaneous cut or editing of genes) compared to other delivery methods available in the art. Non-limiting examples of the various aspects are shown in the present disclosure.

### I. General Techniques

Some of the techniques and procedures described or referenced herein are generally well understood and commonly employed using conventional methodology by those skilled in the art, such as, for example, the widely utilized methodologies described in Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F. M. Ausubel, et al. eds., 2003); the series Methods in Enzymology (Academic Press, Inc.); PCR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6^{th} ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C. A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 2011).

### II. Definitions

For purposes of interpreting this specification, the following definitions will apply and whenever appropriate, terms used in the singular will also include the plural and vice versa. In the event that any definition set forth below conflicts with any document incorporated herein by reference, the definition set forth herein shall control. Additional definitions are set forth throughout the detailed description.

As used herein, the singular form term “a,” “an,” and “the” entity refers to one or more of that entity unless indicated otherwise. As such, the terms “a” (or “an” or “the”), “one or more,” and “at least one” can be used interchangeably herein.

It is understood that aspects and aspects of the disclosure described herein include “comprising,” “consisting,” and “consisting essentially of” aspects and aspects. It is also understood that wherever aspects and aspects are described herein with the language “comprising,” otherwise analogous aspects or aspects described in terms of “consisting of” and/or “consisting essentially of” are also provided.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

For all compositions described herein, and all methods using a composition described herein, the compositions can either comprise the listed components or steps, or can “consist essentially of” the listed components or steps. When a composition is described as “consisting essentially of” the listed components, the composition contains the components listed, and can further contain other components which do not substantially affect the methods disclosed, but do not contain any other components which substantially affect the methods disclosed other than those components expressly listed; or, if the composition does contain extra components other than those listed which substantially affect the methods disclosed, the composition does not contain a sufficient concentration or amount of the extra components to substantially affect the methods disclosed. When a method is described as “consisting essentially of” the listed steps, the method contains the steps listed, and can further contain other steps that do not substantially affect the methods disclosed, but the method does not contain any other steps which substantially affect the methods disclosed other than those steps expressly listed. As a non-limiting specific example, when a composition is described as “consisting essentially of” a component, the composition can additionally contain any amount of pharmaceutically acceptable carriers, vehicles, or diluents and other such components which do not substantially affect the methods disclosed.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “constriction” as used herein refers to a narrowed passageway. In some aspects, the constriction is a microfluidic channel, such as that contained within a microfluidic device. In some aspects, the constriction is a pore or contained within a pore. Where the constriction is a pore, in some aspects, the pore is contained in a surface. Unless indicated otherwise, the term constriction refers to both microfluidic channels and pores, as well as other suitable constrictions available in the art. Therefore, where applicable, disclosures relating to microfluidic channels can also apply to pores and/or other suitable constrictions available in the art. Similarly, where applicable, disclosures relating to pores can equally apply to microfluidic channels and/or other suitable constrictions available in the art.

The term “pore” as used herein refers to an opening, including without limitation, a hole, tear, cavity, aperture, break, gap, or perforation within a material. In some aspects, (where indicated) the term refers to a pore within a surface of a microfluidic device, such as those described in the present disclosure. In some aspects, (where indicated) a pore can refer to a pore in a cell wall and/or cell membrane.

The term “membrane” as used herein refers to a selective barrier or sheet containing pores. The term includes, but is not limited to, a pliable sheet-like structure that acts as a boundary or lining. In some aspects, the term refers to a surface or filter containing pores. This term is distinct from the term “cell membrane,” which refers to a semipermeable membrane surrounding the cytoplasm of cells.

The term “filter” as used herein refers to a porous article that allows selective passage through the pores. In some aspects, the term refers to a surface or membrane containing pores.

As used herein, the terms “deform” and “deformity” (including derivatives thereof) refer to a physical change in a cell. As described herein, as a cell passes through a constriction (such as those of the present disclosure), it experiences various forces due to the constraining physical environment, including but not limited to mechanical deforming forces and/or shear forces that causes perturbations in the cell membrane. As used herein, a “perturbation” within the cell membrane refers to any opening in the cell membrane that is not present under normal steady state conditions (e.g., no deformation force applied to the cells). Perturbation can comprise a hole, tear, cavity, aperture, pore, break, gap, perforation, or combinations thereof.

The term “heterogeneous” as used herein refers to something which is mixed or not uniform in structure or composition. In some aspects, the term refers to pores having varied sizes, shapes, or distributions within a given surface.

The term “homogeneous” as used herein refers to something which is consistent or uniform in structure or composition throughout. In some aspects, the term refers to pores having consistent sizes, shapes, or distribution within a given surface.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. Additionally, polynucleotides useful for the present disclosure can be in any suitable forms known in the art. For instance, in some aspects, polynucleotide comprises a linear polynucleotide, circular polynucleotide, or both. The backbone of the polynucleotide can comprise sugars and phosphate groups (as can typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P-NH2) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues can contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications can be deliberate, as through site-directed mutagenesis, or can be accidental, such as through mutations of hosts which produce the proteins or errors due to PCR amplification.

As used herein, the term “sequential delivery” refers to the delivery of multiple payloads to a cell, where a first payload is delivered to the cell and then the second (or subsequent) payload is delivered to the cell. In some aspects, the first payload, the second payload, or both the first and second payloads can be delivered to the cell using squeeze processing. For instance, in some aspects, the first payload can be delivered to the cell using squeeze processing, and the second payload can be delivered to the cell using non-squeeze processing (e.g., transfection). In some aspects, the first payload can be delivered to the cell using non-squeeze processing (e.g., transfection), and the second payload can be delivered to the cell using squeeze processing. In some aspects, the first payload can be delivered to the cell using a first squeeze, and then the second payload can be delivered to the cell using a second squeeze (also referred to herein as “sequential squeeze” or “sequential squeeze processing”). Accordingly, sequential delivery useful for the present disclosure can comprise multiple squeeze processings. In some aspects, each of the multiple squeeze processings delivers a separate payload to the cell. In some aspects, one or more of the multiple squeeze processings do not involve the delivery of a payload. For instance, in some aspects, a sequential delivery method described herein comprises a first squeeze, a second squeeze, and a third squeeze, wherein the first squeeze comprises passing a cell without any payload through a first constriction, the second squeeze comprises passing the cell from the first squeeze through a second constriction to deliver a first payload to the cell, and the third squeeze comprises passing the cell from the second squeeze through a third constriction to deliver a second payload to the cell. Not to be bound by any one theory, in some aspects, passing the cell through the first constriction without any payload (i.e., the first squeeze) can help prepare the cell for subsequent payload deliveries, e.g., can improve the delivery efficiency of the first payload and/or the second payload.

As used herein, the term “concurrent delivery” (also referred to herein as “co-delivery”) refers to the delivery of multiple payloads to a cell, where the multiple payloads are delivered to the cells at the same time (e.g., as part of a single solution).

### III. Methods of the Disclosure

In some aspects, the present disclosure relates to methods of delivering a cargo (also referred to herein as “payload”) into a cell by passing the cells through a constriction (such as those described herein). As demonstrated herein, as the cells pass through the constriction, they become transiently deformed, such that cell membrane of the cells is perturbed. The perturbations within the cell membrane can allow various payloads to enter or loaded into the cell (e.g., through diffusion). The specific process by which the cells pass through a constriction and become transiently deformed is referred to herein as “squeeze processing” or “squeezing.” As is apparent from the present disclosure, such a delivery method can be used to target a payload to various compartments within a cell. For instance, in some aspects, the squeeze processing methods provided herein can be used to deliver a payload to the cytoplasm of a cell. In some aspects, the delivery methods provided herein are useful for targeting a payload to the nucleus of a cell.

### III.A. Delivering a Payload to the Nucleus

As demonstrated herein, in some aspects, using a plurality of constrictions with a squeeze processing method described herein can allow for the delivery of a payload to a specific compartment of a cell. For instance, the methods described herein can specifically deliver a payload into the nucleus of a cell. Accordingly, in some aspects, provided herein is a method of delivering a payload to the nucleus of a cell, comprising passing a cell suspension, which comprises the cell, through a plurality of constrictions under one or more parameters, wherein passing the cell suspension through the plurality of constrictions allows the payload to enter the cell and be delivered to the nucleus.

As will be apparent to those skilled in the arts, payloads such as nucleic acids (e.g., DNA) or other gene editing tools (e.g., described herein) need to get into the nucleus to exert their activity. Therefore, upon entering the cell, the rapid and specific delivery of the payload to the nucleus can be important. The rapid and specific delivery to the nucleus can also help minimize toxicity induced by cytoplasmic DNA detection by the immune system. In some aspects, with the squeeze processing methods provided herein (e.g., comprising a plurality of constrictions), the delivery of the payload to the nucleus occurs less than about 6 hours, less than about 5 hours, less than about 4 hours, less than about 3 hours, less than about 2 hours, less than about 1 hour, less than about 50 minutes, less than about 40 minutes, less than about 30 minutes, less than about 20 minutes, less than about 10 minutes, less than about 5 minutes, less than about 4 minutes, less than about 3 minutes, less than about 2 minutes, less than about 1 minute, less than about 30 seconds, less than about 20 seconds, less than about 10 seconds, less than about 5 seconds, or less than about 1 second after the payload enters the cell.

Additionally, compared to other methods of delivering a payload to the nucleus of a cell, the methods provided herein allow for greater delivery efficiency of the payload to the nucleus. Accordingly, in some aspects, provided herein is a method of increasing the delivery efficiency of a payload to the nucleus of a cell, comprising passing a cell suspension, which comprises the cell, through a plurality of constrictions under one or more parameters, wherein passing the cell suspension through the plurality of constrictions allows the payload to enter the cell and be delivered to the nucleus with increased delivery efficiency. In some aspects, the delivery efficiency of the payload is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, at least about 50-fold, at least about 60-fold, at least about 70-fold, at least about 80-fold, at least about 90-fold, or at least about 100-fold, compared to a reference delivery efficiency (e.g., the delivery efficiency of the payload to the nucleus of the cell after passing the cells through a single constriction).

As used herein, the term “delivery efficiency” refers to the ability of the payload to be delivered to one or more compartments of a cell. For instance, when used to describe the delivery of a payload to the nucleus of a cell, the term refers to the ability of a payload to traverse the nuclear membrane and enter the nucleus of a cell. When used to describe the delivery of a payload into the cell generally (e.g., cytoplasm), the term can refer to the ability of a payload to traverse the cell membrane and enter the cytoplasm of the cell. Any suitable methods known in the art can be used to measure delivery efficiency as used herein. In some aspects, delivery efficiency is associated with how quickly a payload is able to be delivered to one or more compartments of a cell (e.g., the nucleus) after entering the cell. In some aspects, delivery efficiency is associated with the total amount of a payload that is delivered to one or more compartments of a cell (e.g., the nucleus) of a cell. In some aspects, delivery efficiency is associated with the degree/magnitude of the biological effect that the payload has on the cell. For instance, when the payload comprises a gene editing tool that is capable of reducing the expression of a gene, the delivery efficiency can refer to how much the gene expression is reduced after the delivery of the payload compared to a reference gene expression (e.g., corresponding gene expression in the cell prior to the delivery of the payload).

As further described herein, the squeeze processing methods of the present disclosure have certain distinct properties that are not shared by other delivery methods known in the art. For instance, in addition to the improved ability to deliver various types of payloads into a cell, particularly to the nucleus, the squeeze processing methods described herein exert minimal lasting effects on the cells. Compared to traditional delivery methods such as electroporation, the squeeze processing methods of the present disclosure preserve both the structural and functional integrity of the squeezed cells. Contrary to the delivery methods provided herein, electroporation can induce broad and lasting alterations in gene expression, which can lead to non-specific activation of cells (e.g., human T cells) and delayed proliferation upon antigen stimulation. With the present methods, any alterations to the cells (e.g., perturbations in the cell membrane) is transient and quickly repaired once the cells are removed from the constriction.

Accordingly, in some aspects, the above methods further comprise contacting the cell with the payload prior to passing the cell suspension through the constriction. As is apparent from the present disclosure, contacting the cell with the payload prior to the squeezing can help delivery efficiency, as the payloads would be able to enter the cell as soon as the perturbations in the cell membrane are created through the squeeze processing. For instance, in some aspects, prior to passing the cell suspension through the constriction, the method comprises contacting the cell with the payload to produce the cell suspension. In some aspects, the methods provided herein comprises contacting the cell with the payload as the cell suspension passes through the constriction. In some aspects, the cell is first contacted with the payload during the passing of the cell suspension through the constriction. In some aspects, the cell is in contact with the payload both prior to the passing step (i.e., passing of the cell suspension through the constriction) and during the passing step. In some aspects, the method comprises contacting the cell with the payload after the passing of the cell suspension through the constriction. In some aspects, the cell is first contacted with the payload after the passing of the cell suspension through the constriction. In some aspects, the cell is in contact with the payload prior to, during, and/or, after the passing step. As further described elsewhere in the present disclosure, when the cell is contacted with the payload after the passing step, the contacting occurs soon after the cell has passed through the constriction, such that there are still perturbations within the cell membrane.

As used herein, the “contacting” that can occur between a cell and a payload (e.g., gene editing tool) includes that a cell can be in contact with the payload as long as the payload is capable of entering the cell once there are perturbations within the cell membrane of the cell. To help illustrate, in some aspects, a cell and a payload are in contact if they are both present within the same cell suspension.

### III.B. Delivery of Multiple Payloads (“Multiplex”)

### III.B.1. Concurrent Delivery

As described herein, the squeeze processing methods of the present disclosure can be used to deliver multiple (e.g., two or more) payloads to a cell. In some aspects, the multiple payloads can be delivered to the cells concurrently (e.g., co-delivery). Accordingly, in some aspects, the present disclosure provides a method of concurrently delivering multiple payloads to a cell, comprising passing a cell suspension, which comprises the cell, through a constriction under one or more parameters, wherein passing the cell suspension through the constriction allows the multiple payloads to enter the cells.

In some aspects, the cell is in contact with the multiple payloads prior to passing the cell suspension through the constriction. For instance, in some aspects, prior to passing the cell suspension through the constriction, the cell and the multiple payloads are contacted (e.g., combined in a single solution) to produce the cell suspension. In some aspects, the cell is first contacted with the multiple payloads as the cell suspension passes through the constriction. For example, in some aspects, the constriction can comprise a lumen and an internal surface, wherein the lumen and/or the internal surface can comprise the multiple payloads (e.g., coated on the internal surface or otherwise present within the lumen). Then, as the cell suspension passes through such a constriction, the cell can come into contact with the multiple payloads. In some aspects, the cell is in contact with the multiple payloads both prior to the passing step (i.e., passing of the cell suspension through the constriction) and during the passing step. In some aspects, the cell comes into contact with the multiple payloads after the cell suspension has passed through the constriction (e.g., soon after the cell suspension passes through the constriction such that the perturbation in the cell membrane of the cell is still present). In some aspects, the cell is in contact with the multiple payloads prior to, during, and/or after the passing step.

### III.B.2. Sequential Delivery

In some aspects, the multiple payloads can be delivered to a cell sequentially. For instance, the multiple payloads can comprise a first payload and a second payload, wherein the first and second payloads are delivered to the cell separately. Accordingly, in some aspects, the present disclosure is directed to a method of sequentially delivering a first payload and a second payload to a cell, comprising passing a cell suspension, which comprises the cell, through a first constriction and a second constriction under one or more parameters, wherein passing the cell suspension through the first constriction allows the first payload to enter the cell; and wherein passing the cell suspension through the second constriction allows the second payload to enter the cell. In some aspects, the cell suspension is passed through the second constriction immediately after the cell suspension passes through the first constriction (e.g., within less than about 1 second, e.g., within about 1 μs). In some aspects, after passing through the first constriction, a period of time elapses before the cell suspension is passed through the second constriction. In some aspects, the time between the end of the first constriction (i.e., when the cell suspension has passed through the first constriction) and the beginning of the second constriction (i.e., when the cell suspension begins passing through the second constriction) is at least about 1 minute, at least about 30 minutes, at least about 1 hour, at least about 6 hours, at least about 12 hours, at least about 1 day, at least about 2 days, or at least about 3 days.

As further described elsewhere in the present disclosure, where a plurality of constrictions are used (e.g., first and second constrictions), in some aspects, the plurality of constrictions can be contained within a single device (e.g., microfluidic chip). In some aspects, the plurality of constrictions can be placed in parallel and/or in series within a single microfluidic chip. For instance, with such a microfluidic chip, the cell suspension can be added to the chip one time, and then the cell suspension can sequentially pass through the plurality of constrictions without further intervention.

In some aspects, the plurality of constrictions can be contained within multiple devices (e.g., microfluidic chip), such that each of the multiple devices comprises one or more of the plurality of constrictions. As demonstrated herein, in some aspects, a method of sequential delivery provided herein comprises passing a cell suspension, which comprises a cell, through a first constriction contained in a first microfluidic chip, such that the first payload is able to enter the cell, e.g., through perturbations in the cell membrane caused by the first constriction. In some aspects, the method can further comprise collecting the cell suspension that has passed through the first constriction, and then passing the cell suspension through a second constriction contained in a second microfluidic chip, such that the second payload is able to enter the cell, e.g., through perturbations in the cell membrane caused by the second constriction.

Where the plurality of constrictions are contained within multiple devices, in some aspects, one or more of the plurality of constrictions can be the same. As used herein, “constrictions are the same” where the cell suspension that passed through the first constriction is passed again through the same first constriction (i.e., would now be referred to as a “second constriction”). Constrictions can also be the same where the cell suspension that passed through the first constriction is passed through a second constriction, which has the same properties as the first constriction (e.g., same diameter, length, width, and depth). In some aspects, one or more of the plurality of constrictions are different. As used herein, “constrictions are different” where the constrictions differ in one or more properties (e.g., diameter, length, width, and/or depth). Suitable constrictions that can be used with the above methods are described elsewhere in the present application.

Additionally, where multiple payloads are sequentially delivered to a cell using the squeeze processing methods described herein, one or more of the multiple payloads can be in contact with the cell prior to, during, and/or after the passing step (i.e., passing of the cell suspension through the constriction). In some aspects, the cell is not in contact with the multiple payloads at the same time. For instance, in some aspects, when a cell suspension is passed through a first constriction, the cell is in contact with the first payload but not the second payload. Similarly, in some aspects, when a cell suspension is passed through a second constriction, the cell is in contact with the second payload but not the first payload.

As is apparent from the present disclosure, in some aspects, the cell can be in contact with the multiple payloads at the same time. For instance, in some aspects, when a cell suspension is passed through the first constriction and/or the second constriction, the cell is in contact with both the first and second payloads. For such aspects, one or more delivery parameters under which the cell suspension is passed through the constrictions are different, such that when the cell suspension is passed through the first constriction, only the first payload is capable of entering the cell, and when the cell suspension is passed through the second constriction, only the second payload is capable of entering the cell. Non-limiting examples of such delivery parameters are described elsewhere in the present disclosure.

### III.C. Gene Editing

As is apparent from the present disclosure, in some aspects, the delivery methods provided herein can be particularly useful in the context of gene therapy, where the efficient delivery of one or more gene editing tools to the nucleus of a cell can be critical. Accordingly, in some aspects, the present disclosure is related to a method of modulating the expression of a gene in a cell, comprising passing a cell suspension, which comprises the cell, through a plurality of constrictions under one or more parameters, wherein passing the cell suspension through the plurality of constrictions allows the delivery of one or more payloads to the nucleus of the cell, wherein the one or more payloads are capable of modulating the expression of the gene (also referred to herein as “gene-editing payload”).

In some aspects, delivery of the gene-editing payload to the nucleus of the cell can reduce the expression of the gene in the cell. In some aspects, the expression of the gene is reduced by at least about 5%, at least about 10%, at least about 20%, at least about 30%, at least about 40%, at least about 50%, at least about 60%, at least about 70%, at least about 80%, at least about 90% or more after the delivery of the gene-editing payload to the nucleus of the cell. In some aspects, delivery of the gene-editing payload to the nucleus of the cell can increase the expression of the gene in the cell. In some aspects, the expression of the gene is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold or more after the delivery of the gene-editing payload to the nucleus of the cell.

As described herein, in some aspects, the delivery methods provided herein (e.g., passing a cell suspension through a plurality of constrictions) can target multiple (e.g., two or more) gene-editing payloads to the nucleus of a cell. In some aspects, the multiple gene-editing payloads target the same gene. In such aspects, delivering multiple gene-editing payloads that target the same gene to the nucleus can enhance/increase the modulation of the gene expression compared to a reference gene expression. In some aspects, the reference gene expression is the corresponding gene expression observed in an untreated cell (e.g., not having undergone squeeze processing with the plurality of constrictions). In some aspects, the reference gene expression is the corresponding gene expression observed in a cell that underwent squeeze processing with the plurality of constrictions but without any payload (also referred to herein as “squeeze alone”). In some aspects, the reference gene expression is the corresponding gene expression observed in a cell that was delivered a single gene-editing payload. In some aspects, the modulation of the gene expression is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold or more compared to the reference gene expression.

In some aspects, one or more of the multiple gene-editing payloads target a different gene. Accordingly, in some aspects, by delivering multiple gene-editing payloads to a cell, the expression of multiple genes can be modulated. In some aspects, the expression of the multiple genes are all reduced. In some aspects, the expression of the multiple genes are all increased. In some aspects, the expression of some of the multiple genes are reduced, while the expression of some of the multiple genes are increased.

Suitable gene-editing payloads are further described elsewhere in the present disclosure. In some aspects, multiple gene-editing payloads comprise at least about 2, at least about 3, at least about 4, at least about 5, at least about 6, at least about 7, at least about 8, at least about 9, or at least about 10 types of payloads. In some aspects, each of the multiple payloads are different.

As will be apparent to those skilled in the arts, the gene editing methods described herein (e.g., passing a cell suspension comprising the gene-editing payload through a plurality of constrictions) can be used to modulate the expression of any suitable genes known in the art. For instance, in some aspects, the gene editing methods provided herein can be used to decrease the expression of a gene associated with a disease or disorder. In some aspects, the gene editing methods can decrease the expression of a gene associated with an impaired cell function. Non-limiting examples of suitable genes that can be regulated include a beta-2 microglobulin (B2M), a T-cell immunoglobulin and mucin-domain containing-3 (TIM3), a T-cell receptor alpha constant (TRAC), CD86, TGF-β, PD-1, BC11a, CCR5, CD38, CISH, or combinations thereof.

The gene editing activity of the methods provided above can be assessed (e.g., quanitified) using any suitable methods known in the art. For instance, in some aspects, after the delivery of a gene-editing payload to the nucleus of a cell, the expression of the protein associated with the target gene can be measured, e.g., using flow cytometry. Non-limiting examples of other methods that can be used include qPCR, Sanger sequencing (e.g., with Tracking of Indels by Decomposition (TIDE)), 10× genomic sequencing, next-generation sequencing (NGS) (e.g., Illumina), long read sequencing (e.g., PacBio and Oxford Nanopore), UDITAS™, tagmentation, GUIDE-Seq, CIRCLE-Seq, T7 endonuclease, and combinations thereof.

### III.D. Cell Suspensions

In some aspects, a cell suspension described herein comprises any suitable cells known in the art that can be modified (e.g., by introducing a payload, e.g., gene-editing tool) using the squeeze processing methods described herein.

In some aspects, the cells are stem cells. As used herein, the term “stem cells” refer to cells having not only self-replication ability but also the ability to differentiate into other types of cells. In some aspects, stem cells useful for the present disclosure comprise induced pluripotent stem cells (iPSCs), embryonic stem cells (ESCs), tissue-specific stem cells (e.g., liver stem cells, cardiac stem cells, or neural stem cells), mesenchymal stem cells, hematopoietic stem cells (HSCs), or combinations thereof.

In some aspects, the cells are somatic cells. As used herein, the term “somatic cells” refer to any cell in the body that are not gametes (sperm or egg), germ cells (cells that go on to become gametes), or stem cells. Non-limiting examples of somatic cells include blood cells, bone cells, muscle cells, nerve cells, or combinations thereof. In some aspects, somatic cells useful for the present disclosure comprise blood cells. In some aspects, the blood cells are peripheral blood mononuclear cells (PBMCs). As used herein, “PBMCs” refer to any peripheral blood cells having a round nucleus. In some aspects, PBMCs comprise an immune cell. As used herein the term “immune cell” refers to any cell that plays a role in immune function. In some aspects, immune cell comprises a T cell, B cell, natural killer (NK) cell, dendritic cell (DC), NKT cell, mast cell, monocyte, macrophage, basophil, eosinophil, neutrophil, DC2.4 dendritic cell, or combinations thereof. In some aspects, the immune cell is a T cell (e.g., human T cell). In some aspects, the immune cell is a B cell. In some aspects, the immune cell is a NK cell. In some aspects, the immune cell is a DC (e.g., DC2.4 dendritic cell). In some aspects, the immune cell is a NKT cell. In some aspects, the immune cell is a mast cell. In some aspects, the immune cell is a monocyte. In some aspects, the immune cell is a macrophage. In some aspects, the immune cell is a basophil. In some aspects, the immune cell is an eosinophil. In some aspects, the immune cell is a neutrophil. In some aspects, the blood cells are red blood cells. In some aspects, the cell is a cancer cell. In some aspects, the cancer cell is a cancer cell line cell, such as a HeLa cell. In some aspects, the cancer cell is a tumor cell. In some aspects, the cancer cell is a circulating tumor cell (CTC). In some aspects, the cell is a fibroblast cell, such as a primary fibroblast or newborn human foreskin fibroblast (Nuff cell). In some aspects, the cell is an immortalized cell line cell, such as a HEK293 cell or a CHO cell. In some aspects, the cell is a skin cell. In some aspects, the cell is a reproductive cell such as an oocyte, ovum, or zygote. In some aspects, the cell is a cluster of cells, such as an embryo, given that the cluster of cells is not disrupted when passing through the pore.

In some aspects, the cell suspension useful for the present disclosure comprises a mixed or purified population of cells. In some aspects, the cell suspension is a mixed cell population, such as whole blood, lymph, PBMCs, or combinations thereof. In some aspects, the cell suspension is a purified cell population. In some aspects, the cell is a primary cell or a cell line cell.

As demonstrated herein, the delivery of a payload (e.g., gene-editing payloads) into a cell can be regulated through one or more parameters of the process in which a cell suspension is passed through a constriction. In some aspects, the specific characteristics of the cell suspension can impact the delivery of a payload into a cell. Such characteristics include, but are not limited to, osmolarity, salt concentration, serum content, cell concentration, pH, temperature or combinations thereof.

In some aspects, the cell suspension comprises a homogeneous population of cells. In some aspects, the cell suspension comprises a heterogeneous population of cells (e.g., whole blood or a mixture of cells in a physiological saline solution or physiological medium other than blood). In some aspects, the cell suspension comprises an aqueous solution. In some aspects, the aqueous solution comprises a cell culture medium, PBS, salts, sugars, growth factors, animal derived products, bulking materials, surfactants, lubricants, vitamins, polypeptides, an agent that impacts actin polymerization, or combinations thereof. In some aspects, the cell culture medium comprises DMEM, OptiMEM, EVIDM, RPMI, or combinations thereof. Additionally, solution buffer can include one or more lubricants (pluronics or other surfactants) that can be designed to reduce or eliminate clogging of the surface and improve cell viability. Exemplary surfactants include, without limitation, poloxamer, polysorbates, sugars such as mannitol, animal derived serum, and albumin protein.

When the suspension includes certain types of cells, in some aspects, the cells can be treated with a solution that aids in the delivery of the payload (e.g., gene-editing payload) to the interior of the cell. In some aspects, the solution comprises an agent that impacts actin polymerization. In some aspects, the agent that impacts actin polymerization comprises Latrunculin A, Cytochalasin, Colchicine, or combinations thereof. For example, in some aspects, the cells can be incubated in a depolymerization solution, such as Lantrunculin A, for about 1 hour prior to passing the cells through a constriction to depolymerize the actin cytoskeleton. In some aspects, the cells can be incubated in Colchicine (Sigma) for about 2 hours prior to passing the cells through a constriction to depolymerize the microtubule network.

In some aspects, a characteristic of a cell suspension that can affect the delivery of a payload (e.g., gene-editing payload) into a cell is the viscosity of the cell suspension. As used herein, the term “viscosity” refers to the internal resistance to flow exhibited by a fluid. In some aspects, the viscosity of the cell suspension is between about 8.9×10-4 Pa·s to about 4.0×10-3 Pa·s, between about 8.9×10-4 Pa·s to about 3.0×10-3 Pas, between about 8.9×10-4 Pas to about 2.0×10-3 Pas, or between about 8.9×10-4 Pa·s to about 1.0×10-3 Pas. In some aspects, the viscosity is between about 0.89 cP to about 4.0 cP, between about 0.89 cP to about 3.0 cP, between about 0.89 cP to about 2.0 cP, or between about 0.89 cP to about 1.0 cP. In some aspects, a shear thinning effect is observed, in which the viscosity of the cell suspension decreases under conditions of shear strain. Viscosity can be measured by any suitable method known in the art, including without limitation, viscometers, such as a glass capillary viscometer or rheometers. A viscometer measures viscosity under one flow condition, while a rheometer is used to measure viscosities which vary with flow conditions. In some aspects, the viscosity is measured for a shear thinning solution such as blood. In some aspects, the viscosity is measured between about 0° C. and about 45° C. For example, the viscosity of the cell suspension can be measured at room temperature (e.g., about 20° C.), physiological temperature (e.g., about 37° C.), higher than physiological temperature (e.g., greater than about 37° C. to about 45° C. or more), reduced temperature (e.g., about 0° ° C. to about 4ºC), or temperatures between these exemplary temperatures.

### III. E. Payloads

As described herein, in some aspects, a cell suspension additionally comprises one or more payloads (e.g., gene-editing payload). The payloads can be present in the cell suspension prior to, during, and/or after the passing step, in which the cell suspension is passed through the constriction. In some aspects, the cell suspension comprises at least about 2, at least about 3, at least about 4, at least about 5, at least about 6, at least about 7, at least about 8, at least about 9, or at least about 10 or more payloads. As further described elsewhere in the present disclosure, in some aspects, a cell suspension can be passed through multiple constrictions. In such aspects, a payload can be loaded into a cell when the cells pass through one or more of the multiple constrictions. In some aspects, a payload is loaded into a cell each time the cells pass through one or more of the multiple constrictions. When multiple payloads are involved, each of the payloads can be the same. In some aspects, one or more of the payloads are different.

As is apparent from the present disclosure, any suitable payloads known in the art can be delivered to a cell using the methods described herein. Non-limiting examples of suitable payloads include a nucleic acid, a polypeptide, a lipid, a carbohydrate, a small molecule, a metal-containing compound, an antibody, a transcription factor, a nanoparticle, a liposome, a fluorescently tagged molecule, or combinations thereof. In some aspects, the nucleic acid comprises a DNA, RNA, or both. In some aspects, DNA comprises a recombinant DNA, a cDNA, a genomic DNA, or combinations thereof. In some aspects, RNA comprises a siRNA, a mRNA, a miRNA, a lncRNA, a tRNA, a shRNA, a self-amplifying mRNA, or combinations thereof. In some aspects, the RNA is mRNA. In some aspects, the RNA is siRNA. In some aspects, the RNA is shRNA. In some aspects, the RNA is miRNA. In some aspects, a small molecule comprises an impermeable small molecule. As used herein, an “impermeable small molecule” refers to a small molecule that naturally does not cross the cell membrane of a cell. In some aspects, the payload comprises a complex of two or more different types of payloads. For example, in some aspects, the payload comprises a protein-nucleic acid complex. In some aspects, the protein-nucleic acid complex comprises a ribonucleoprotein and a mRNA. In some aspects, the protein-nucleic acid complex comprises a nucleic acid molecule that is complexed with a protein, e.g., via electrostatic attraction, a nucleic acid molecule wrapped around a protein; DNA and a histone (nucleosome); a ribonucleoprotein (RNP); a ribosome; an enzyme telomerase; a vault ribonucleoprotein; ribonuclease (RNase) (e.g., RNase P); heterogeneous ribonucleoprotein particle (hnRNP); a small nuclear RNP (snRNP); a chromosome comprising a protein; or combinations thereof.

In some aspects, the delivery methods described herein can be used to deliver a wide range of polypeptide complexes to a cell. Non-limiting examples of such complexes include a proteasome, a holoenzyme, an RNA polymerase, a DNA polymerase, a spliceosome, a vault cytoplasmic ribonucleoprotein, a small nuclear ribonucleic protein (snRNP), a telomerase, a nucleosome, a death signaling complex (DISC), a mammalian target of rapamycin complex 1 (mTORC1), a mammalian target of rapamycin complex 2 (mTORC2), or a class I phosphoinositide 3 kinase (Class I PI3K), histone-DNA complex, toll-like receptor (TLR)-agonist complex, transposase/transposon complex, tRNA ribosome complex, polypeptide-protease complex, an enzyme-substrate complex, or combinations thereof.

In some aspects, a payload that can be delivered to a cell using the methods described herein comprises a ribonucleoprotein complex. In some aspects, the ribonucleoprotein complex comprises a RNA-induced silencing complex (RISC). “RISC” is a catalytically active protein-RNA complex that is an important mediator of RNA interference (RNAi). RISC incorporates a strand of a double-stranded RNA (dsRNA) fragment, such as small interfering RNA (siRNA) or microRNA (miRNA). The strand acts as a template for RISC to recognize a complementary messenger RNA (mRNA) transcript. Argonaute, a protein component of RISC, subsequently activates and cleaves the mRNA.

In some aspects, a ribonucleoprotein complex comprises a ribosome. “Ribosomes” consist of small and large ribosomal subunits, with each subunit composed of one or more ribosomal RNA (rRNA) molecules and a variety of proteins. Together, the ribosome complex mediates the translation of mRNA into polypeptide.

In some aspects, a payload that can be delivered to a cell comprises a transposase bound to a target DNA. In some aspects, the transposase enzyme-target DNA complexes are delivered to mediate nucleic acid integration of the target DNA into the cell.

In some aspects, a payload that is useful for the present disclosure comprises a transcription factor complex. In some aspects, the transcription factor complex consists of a transcription factor bound to a preinitiation complex, a large complex of proteins and RNA polymerase which is necessary for modulating gene transcription.

In some aspects, a payload that can be used with the present disclosure comprises a gene-editing payload (also referred to herein as “gene editing tool”). Any suitable gene editing tools known in the art can be used with the present disclosure. Non-limiting examples of such gene editing tools include a shRNA, siRNA, miRNA, antisense oligonucleotides, zinc-finger nuclease, meganuclease, transcription activator-like effector nuclease (TALEN), a CRISPR/Cas system, a ribonucleoprotein (RNP), a Cre recombinase, or any combination thereof. In some aspects, the gene editing tool is a CRISPR/Cas system. In some aspects, the CRISPR/Cas system comprises a Cas9 nuclease.

Additional disclosures relating to exemplary payloads that can be delivered to a cell are provided below:

### III.E.1. CRISPR/Cas System

In some aspects, the gene editing tool that can be used in the present disclosure comprises a CRISPR/Cas system. Such systems can employ, for example, a Cas9 nuclease, which in some instances, is codon-optimized for the desired cell type in which it is to be expressed (e.g., T cells). CRISPR/Cas systems use Cas nucleases, e.g., Cas9 nucleases, that are targeted to a genomic site by complexing with a synthetic guide RNA (gRNA) that hybridizes to a target DNA sequence immediately preceding a protospacer adjacent motif (PAM) site, e.g., an NGG motif recognized by the Cas nuclease, e.g., Cas9. This results in a double-strand break three nucleotides upstream of the NGG motif. In some aspects, the break can have sticky ends. In some aspects, a modified version of a Cas nuclease (e.g., Cas9 nickase) can be used that will lead to a single stranded nick as opposed to a double stranded break. Additional fusions with other enzymes can lead to site-specific base editing in the absence of a double stranded break. A unique capability of the CRISPR/Cas9 system is the ability to simultaneously target multiple distinct genomic loci by co-expressing a single Cas9 protein with two or more gRNAs (e.g., at least one, two, three, four, five, six, seven, eight, nine or ten gRNAs). Such systems can also employ a guide RNA (gRNA) that comprises two separate molecules. In some aspects, the two-molecule gRNA comprises a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA” or “scaffold”) molecule.

A crRNA comprises both the DNA-targeting segment (single stranded) of the gRNA and a stretch of nucleotides that forms one half of a double stranded RNA (dsRNA) duplex of the protein-binding segment of the gRNA. A corresponding tracrRNA (activator-RNA) comprises a stretch of nucleotides that forms the other half of the dsRNA duplex of the protein-binding segment of the gRNA. Thus, a stretch of nucleotides of a crRNA is complementary to and hybridizes with a stretch of nucleotides of a tracrRNA to form the dsRNA duplex of the protein-binding domain of the gRNA. As such, each crRNA can be said to have a corresponding tracrRNA. The crRNA additionally provides the single stranded DNA-targeting segment. Accordingly, a gRNA comprises a sequence that hybridizes to a target sequence and a tracrRNA. Thus, a crRNA and a tracrRNA (as a corresponding pair) hybridize to form a gRNA. If used for modification within a cell, the exact sequence and/or length of a given crRNA or tracrRNA molecule can be designed to be specific to the species in which the RNA molecules will be used (e.g., humans).

Naturally-occurring genes encoding the three elements (Cas9, tracrRNA and crRNA) are typically organized in operon(s). Naturally-occurring CRISPR RNAs differ depending on the Cas9 system and organism but often contain a targeting segment of between 21 to 72 nucleotides length, flanked by two direct repeats (DR) of a length of between 21 to 46 nucleotides (see, e.g., WO2014/131833). In the case of S. pyogenes, the DRs are 36 nucleotides long and the targeting segment is 30 nucleotides long. The 3′ located DR is complementary to and hybridizes with the corresponding tracrRNA, which in turn binds to the Cas9 protein.

Alternatively, a CRISPR system used herein can further employ a fused crRNA-tracrRNA construct (i.e., a single transcript) that functions with the codon-optimized Cas9. This single RNA is often referred to as a guide RNA or gRNA or single guide RNA, or sgRNA. Within a gRNA, the crRNA portion is identified as the “target sequence” for the given recognition site and the tracrRNA is often referred to as the “scaffold.” Briefly, a short DNA fragment containing the target sequence is inserted into a guide RNA expression plasmid. The gRNA expression plasmid comprises the target sequence (in some aspects around 20 nucleotides), a form of the tracrRNA sequence (the scaffold) as well as a suitable promoter that is active in the cell and necessary elements for proper processing in eukaryotic cells. Many of the systems rely on custom, complementary oligos that are annealed to form a double stranded DNA and then cloned into the gRNA expression plasmid.

The gRNA expression cassette and the Cas9 expression cassette are then introduced into the cell. See, for example, Mali P et al., (2013) Science 2013 Feb. 15; 339(6121):823-6; Jinek Metal., Science 2012 Aug. 17; 337(6096):816-21; Hwang W Y et al., Nat Biotechnol 2013 March; 31(3):227-9; Jiang W et al., Nat Biotechnol 2013 March; 31(3):233-9; Cronican et al., ACS Chem. Biol. 5(8):747-52 (2010); and Cong L et al., Science 2013 Feb. 15; 339(6121):819-23, each of which is herein incorporated by reference in its entirety. See also, for example, WO/2013/176772 A1, WO/2014/065596 A1, WO/2014/089290 A1, WO/2014/093622 A2, WO/2014/099750 A2, and WO/2013142578 A1, each of which is herein incorporated by reference in its entirety.

Accordingly, in some aspects, a gene editing tool that can be delivered to the nucleus of a cell comprises a Cas protein. In some aspects, any known Cas protein can be used with the present disclosure. Non-limiting examples of Cas proteins that are useful for the present disclosure include: Cas1, Cas2, Cas3, Cas4, Cas5, Cas6, Cas7, Cas8, Cas9, Cas12, Cas13, CasX, CasY, and combinations thereof. In some aspects, a Cas protein useful for the present disclosure is from Streptococcus pyogenes. In some aspects, Cas protein from other species (e.g., Staphylococcus aureus) can be used with the present disclosure. In some aspects, the Cas protein is a Cas9 nuclease.

In some aspects, a gene-editing payload comprising a Cas protein (e.g., Cas9) can further comprise a guide RNA (gRNA). For instance, in some aspects, a Cas protein (e.g., Cas9) can be delivered to a cell as a complex protein, in which the Cas protein and the gRNA are associated with each other.

In some aspects, the gene editing tool (e.g., Cas) can be introduced into the cell as a protein, which then passes through the nuclear membrane to enter the nucleus. In some aspects, the gene editing tool (e.g., mRNA) can be introduced into the cell as mRNA, which is then translated prior to being delivered to the nucleus of the cell. In some aspects, the Cas protein (or any of the other gene editing tools described herein) can be formulated with a lipid to form lipid nanoparticles (LNPs).

### III.E.2. Meganuclease

In some aspects, the gene editing tool that can be used in the present disclosure comprises a nuclease agent, such as a meganuclease system. Meganucleases have been classified into four families based on conserved sequence motifs, the families are the “LAGLIDADG,” “GIY-YIG,” “H—N—H,” and “His-Cys box” families. These motifs participate in the coordination of metal ions and hydrolysis of phosphodiester bonds.

HEases are notable for their long recognition sites, and for tolerating some sequence polymorphisms in their DNA substrates. Meganuclease domains, structure and function are known, see, for example, Guhan and Muniyappa (2003) Crit Rev Biochem Mol Biol 38:199-248; Lucas et al., (2001) Nucleic Acids Res 29:960-9; Jurica and Stoddard, (1999) Cell Mol Life Sci 55:1304-26; Stoddard, (2006) Q Rev Biophys 38:49-95; and Moure et al., (2002) Nat Struct Biol 9:764; each of which is herein incorporated by reference in its entirety.

In some aspects, a naturally occurring variant, and/or engineered derivative meganuclease can be used. Methods for modifying the kinetics, cofactor interactions, expression, optimal conditions, and/or recognition site specificity, and screening for activity are known, see for example, Epinat et al., (2003) Nucleic Acids Res 31:2952-62; Chevalier et al., (2002) Mol Cell 10:895-905; Gimble et al., (2003) Mol Biol 334:993-1008; Seligman et al., (2002) Nucleic Acids Res 30:3870-9; Sussman et al., (2004) J Mol Biol 342:31-41; Rosen et al., (2006) Nucleic Acids Res 34:4791-800; Chames et al., (2005) Nucleic Acids Res 33:e178; Smith et al., (2006) Nucleic Acids Res 34:e149; Gruen et al., (2002) Nucleic Acids Res 30:e29; Chen and Zhao, (2005) Nucleic Acids Res 33:e154; WO2005105989; WO2003078619; WO2006097854; WO2006097853; WO2006097784; and WO2004031346; each of which is herein incorporated by reference in its entirety.

Any meganuclease can be used herein, including, but not limited to, I-Scel, I-Scell, I-SceIII, I-SceIV, I-SceV, I-SecVI, I-SceVII, I-CeuI, I-CeuAIIP, I-Crel, I-CrepsbIP, I-CrepsbIIP, I-CrepsbIIIP, I-CrepsbIVP, I-TliI, I-PpoI, PI-PspI, F-Scel, F-SceII, F-SuvI, F-TevI, F-TevII, I-Amal, I-AniI, I-Chul, I-Cmoel, I-Cpal, I-CpaII, I-CsmI, I-Cvul, I-CvuAIP, I-Ddil, I-DdiII, I-DirI, I-DmoI, I-HmuI, I-HmuII, I-HsNIP, I-LlaI, I-Msol, I-Naal, I-NanI, I-NcIIP, I-NgrIP, I-NitI, I-Njal, I-Nsp236IP, I-PakI, I-PboIP, I-PcuIP, I-PcuAI, I-PcuVI, I-PgrIP, I-PobIP, I-PorIIP, I-PbpIP, I-SpBetaIP, I-Scal, I-SexIP, I-SneIP, I-SpomI, I-SpomCP, I-SpomIP, I-SpomIIP, I-SquIP, I-Ssp68031, I-SthPhiJP, I-SthPhiST3P, I-SthPhiSTe3bP, I-TdeIP, I-TevI, I-TevII, I-TevIII, I-UarAP, I-UarHGPAIP, I-UarHGPA13P, I-VinIP, I-ZbiIP, PI-Mtul, PI-MtuHIP, PI-MtuHIIP, PI-PfuI, PI-PfuII, PI-PkoI, PI-PkoII, PI-Rma43812IP, PI-SpBetaIP, PI-Scel, PI-Tful, PI-TfuII, PI-ThyI, PI-TliI, PI-TliII, or any active variants or fragments thereof.

### III.E.3. TALEN

In some aspects, the gene editing tool that can be delivered to the nucleus of a cell using the methods described herein comprises a nuclease agent, such as a Transcription Activator-Like Effector Nuclease (TALEN). TAL effector nucleases are a class of sequence-specific nucleases that can be used to make double-strand breaks at specific target sequences in the genome of a prokaryotic or eukaryotic organism. TAL effector nucleases are created by fusing a native or engineered transcription activator-like (TAL) effector, or functional part thereof, to the catalytic domain of an endonuclease, such as, for example, FokI.

The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al., (2010) PNAS 10.1073/pnas. 1013133107; Scholze & Boch (2010) Virulence 1:428-432; Christian et al., Genetics (2010) 186:757-761; Li et al., (2010) Nuc. Acids Res. (2010) doi: 10.1093/nar/gkq704; and Miller et al., (2011) Nature Biotechnology 29:143-148; all of which are herein incorporated by reference in their entirety.

Non-limiting examples of suitable TAL nucleases, and methods for preparing suitable TAL nucleases, are disclosed, e.g., in US Patent Application Nos. 2011/0239315 A1, 2011/0269234 A1, 2011/0145940 A1, 2003/0232410 A1, 2005/0208489 A1, 2005/0026157 A1, 2005/0064474 A1, 2006/0188987 A1, and 2006/0063231 A1, each of which is herein incorporated by reference in its entirety.

In some aspects, TAL effector nucleases are engineered that cut in or near a target nucleic acid sequence in, e.g., a genomic locus of interest, wherein the target nucleic acid sequence is at or near a sequence to be modified by a targeting vector. The TAL nucleases suitable for use with the various methods and compositions provided herein include those that are specifically designed to bind at or near target nucleic acid sequences to be modified by targeting vectors as described herein.

### III.E.4. Zinc-Finger Nuclease (ZFN)

In some aspects, a gene editing tool that can be delivered to the nucleus of a cell using the methods described herein comprises a nuclease agent, such as a zinc-finger nuclease (ZFN) system. Zinc finger-based systems comprise a fusion protein comprising two protein domains: a zinc finger DNA binding domain and an enzymatic domain. A “zinc finger DNA binding domain”, “zinc finger protein”, or “ZFP” is a protein, or a domain within a larger protein, that binds DNA in a sequence-specific manner through one or more zinc fingers, which are regions of amino acid sequence within the binding domain whose structure is stabilized through coordination of a zinc ion. The zinc finger domain, by binding to a target DNA sequence, directs the activity of the enzymatic domain to the vicinity of the sequence and, hence, induces modification of the endogenous target gene in the vicinity of the target sequence. A zinc finger domain can be engineered to bind to virtually any desired sequence. Accordingly, after identifying a target genetic locus containing a target DNA sequence at which cleavage or recombination is desired, one or more zinc finger binding domains can be engineered to bind to one or more target DNA sequences in the target genetic locus. Expression of a fusion protein comprising a zinc finger binding domain and an enzymatic domain in a cell, effects modification in the target genetic locus.

In some aspects, a zinc finger binding domain comprises one or more zinc fingers. See, e.g., Miller et al., (1985) EMBO J. 4:1609-1614; Rhodes (1993) Scientific American February:56-65; U.S. Pat. No. 6,453,242; each of which is herein incorporated by reference in its entirety. Typically, a single zinc finger domain is about 30 amino acids in length. An individual zinc finger binds to a three-nucleotide (i.e., triplet) sequence (or a four-nucleotide sequence which can overlap, by one nucleotide, with the four-nucleotide binding site of an adjacent zinc finger). Therefore, the length of a sequence to which a zinc finger binding domain is engineered to bind (e.g., a target sequence) will determine the number of zinc fingers in an engineered zinc finger binding domain. For example, for ZFPs in which the finger motifs do not bind to overlapping subsites, a six-nucleotide target sequence is bound by a two-finger binding domain; a nine-nucleotide target sequence is bound by a three-finger binding domain, etc. Binding sites for individual zinc fingers (i.e., subsites) in a target site need not be contiguous, but can be separated by one or several nucleotides, depending on the length and nature of the amino acids sequences between the zinc fingers (i.e., the inter-finger linkers) in a multi-finger binding domain. In some aspects, the DNA-binding domains of individual ZFNs comprise between three and six individual zinc finger repeats and can each recognize between 9 and 18 basepairs.

Zinc finger binding domains can be engineered to bind to a sequence of choice. See, for example, Beerli et al., (2002) Nature Biotechnol. 20:135-141; Pabo et al., (2001) Ann. Rev. Biochem. 70:313-340; Isalan et al., (2001) Nature Biotechnol. 19:656-660; Segal et al., (2001) Curr. Opin. Biotechnol. 12:632-637; Choo et al., (2000) Curr. Opin. Struct. Biol. 10:411-416; 2002-2003 Catalogue, New England Biolabs, Beverly, Mass.; and Belfort et al., (1997) Nucleic Acids Res. 25:3379-3388; each of which is herein incorporated by reference in its entirety. An engineered zinc finger binding domain can have a novel binding specificity, compared to a naturally-occurring zinc finger protein. Engineering methods include, but are not limited to, rational design and various types of selection.

Exemplary restriction endonucleases (restriction enzymes) suitable for use as an enzymatic domain of the ZFPs described herein are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al., (1992) Proc. Natl. Acad. Sci. USA 89:4275-4279; Li et al., (1993) Proc. Natl. Acad. Sci. USA 90:2764-2768; Kim et al., (1994a) Proc. Natl. Acad. Sci. USA 91:883-887; Kim et al., (1994b) J. Biol. Chem. 269: 31,978-31,982, each of which is herein incorporated by reference in its entirety.

### III.E.5. Interference RNA (RNAi)

In some aspects, a gene editing tool that can be delivered to the nucleus of a cell comprises an RNA intereference molecule (RNAi). As used herein, RNAi are RNA polynucleotide that mediates the decreased the expression of an endogenous target gene product by degradation of a target mRNA through endogenous gene silencing pathways (e.g., Dicer and RNA-induced silencing complex (RISC)). Non-limiting examples of RNAi agents include micro RNAs (also referred to herein as “miRNAs”), short hair-pin RNAs (shRNAs), small interfering RNAs (siRNAs), RNA aptamers, or combinations thereof.

In some aspects, the gene editing tools useful for the present disclosure comprises one or more miRNAs. “miRNAs” refer to naturally occurring, small non-coding RNA molecules of about 21-25 nucleotides in length. miRNAs can downregulate (e.g., decrease) expression of an endogenous target gene product through translational repression, cleavage of the mRNA, and/or deadenylation. Non-limiting examples of miRNAs that can be used with the present disclosure include: miR-103a, miR-106a, miR-106b, miR-107, miR-10a, miR-126, miR-1260a, miR-1260b, miR-1280, miR-128, miR-130b, miR-148a, miR-151a, miR-15b, miR-16, miR-17, miR-181a, miR-182, miR-183, miR-186, miR-18a, miR-18b, miR-191, miR-192, miR-19a, miR-19b, miR-20a, miR-20b, miR-21, miR-221, miR-25, miR-26a, miR-27b, miR-28, miR-30a, miR-30b, miR-30c, miR-30d, miR-30e, miR-320a, miR-320b, miR-320c, miR-378a, miR-486, miR-92a, miR-92b, miR-93, miR-99b, miR-FF4, miR-FF5, miR-451, or combinations thereof.

In some aspects, a gene editing tool that can be used with the present disclosure comprises one or more shRNAs. “shRNAs” (or “short hairpin RNA” molecules) refer to an RNA sequence comprising a double-stranded region and a loop region at one end forming a hairpin loop, which can be used to reduce and/or silence a gene expression. The double-stranded region is typically about 19 nucleotides to about 29 nucleotides in length on each side of the stem, and the loop region is typically about three to about ten nucleotides in length (and 3′- or 5′-terminal single-stranded overhanging nucleotides are optional). shRNAs can be cloned into plasmids or in non-replicating recombinant viral vectors to be introduced intracellularly and result in the integration of the shRNA-encoding sequence into the genome. As such, an shRNA can provide stable and consistent repression of endogenous target gene translation and expression.

In some aspects, a gene editing tool useful for the present disclosure comprises one or more siRNAs. “siRNAs” refer to double stranded RNA molecules typically about 21-23 nucleotides in length. The siRNA associates with a multi protein complex called the RNA-induced silencing complex (RISC), during which the “passenger” sense strand is enzymatically cleaved. The antisense “guide” strand contained in the activated RISC then guides the RISC to the corresponding mRNA because of sequence homology and the same nuclease cuts the target mRNA, resulting in specific gene silencing. In some aspects, an siRNA is about 18, about 19, about 20, about 21, about 22, about 23, or about 24 nucleotides in length and has a 2 base overhang at its 3′ end. siRNAs and shRNAs are further described in Fire et al., Nature 391:19, 1998 and U.S. Pat. Nos. 7,732,417; 8,202,846; and 8,383,599; each of which is herein incorporated by reference in its entirety.

### III.E.6. Base Editors

In some aspects, the gene editing tool that can be used in the present disclosure comprises a base editor. “Base editors” refer to engineered ribonucleoprotein complexes that act as tools for base editing in cells and organism. “Base editing” is the conversion of one target base or base pair into another (e.g. A:T to G:C, C:G to T:A) without requiring the creation and repair of double-stranded breaks (DSB). Non-limiting examples of base editors that can be delivered to the nucleus of a cell using the methods disclosed herein include: cytosine base editors (CBEs), adenine base editors (ABEs), prime editors, and combinations thereof. See, e.g., US Publ. No. 2020/0063114 A1, which is incorporated herein by reference in its entirety.

As disclosed herein, the above examples of gene editing tools are not intended to be limiting and any gene editing tool available in the art can be used with the present disclosure.

In some aspects, the squeeze processing methods described herein can be used to deliver additional compounds, e.g., in combination with the payloads described above (e.g., gene-editing payload). As described elsewhere herein, the squeeze processing method of the present disclosure differs from the more traditional approaches to delivering payloads into cells. With the use of viral vectors (e.g., AAV or lentivirus) or with electroporation/lipofection, there are often cytotoxicity and/or homogeneity issues that make such approaches less desirable. With the present methods, as demonstrated herein, there are no lasting negative effects on the cells (e.g., majority of the squeeze-processed cells remain viable and resemble their non-squeeze-processed counterparts). Also, in some aspects, additional compounds can be delivered to cells using the present methods, wherein the additional compounds help improve one or more properties of a mixture comprising the payload (e.g., gene-editing payload). In some aspects, the additional compound can comprise a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

### III.E.7. Lipid Formulations

In some aspects, any of the payloads described herein can be formulated with one or more lipids to form a lipid nanoparticle (LNP) formulation. Non-limiting examples of lipids that can be used are described in, e.g., US20190136231A1 and US20200392541A1, each of which is incorporated herein by reference in its entirety. As used herein, “lipid nanoparticle” or “LNP” refers to a particle that comprises a plurality of lipid molecules physically associated with each other by intermolecular forces. The LNPs can be, e.g., microspheres (including unilamellar and multilamellar vesicles, e.g., “liposomes”—lamellar phase lipid bilayers that, in some aspects, are substantially spherical—and, in some aspects, can comprise an aqueous core, e.g., comprising a substantial portion of RNA molecules), a dispersed phase in an emulsion, micelles, or an internal phase in a suspension.

In some aspects, a suitable lipid is Lipid A, which is (9Z,12Z)-3-((4,4-bis(octyloxy)butanoyl)oxy)-2-((((3-(diethylamino)propoxy)carbonyl)oxy)methyl)propyl octadeca-9,12-dienoate; also called 3-((4,44ois(octyloxy)butanoyl)oxy)-2-((((3-(diethylamino)propoxy)carbonyl)oxy)methyl)propyl (9Z,12Z)-octadeca-9,12-dienoate. In some aspects, a suitable lipid is Lipid B, which is ((5-((dimethylamino)methyl)-1,3-phenylene)bis(oxy))bis(octane-8,1-diyl)bis(decanoate); also called ((5-((dimethylamino)methyl)-1,3-phenylene)bis(oxy))bis(octane-8,1-diyl) bis(decanoate). In some aspects, a suitable lipid is Lipid C, which is 2-((4-(((3-(dimethylamino)propoxy)carbonyl)oxy)hexadecanoyl)oxy)propane-1,3-diyl (9Z,9′Z,12Z,12′Z)-bis(octadeca-9,12-dienoate). In some aspects, a suitable lipid is Lipid D, which is 3-(((3-(dimethylamino)propoxy)carbonyl)oxy)-13-(octanoyloxy)tridecyl-3-octylundecanoate.

Non-limiting examples of other suitable lipids include 5-heptadecylbenzene-1,3-diol (resorcinol), dipalmitoylphosphatidylcholine (DPPC), distearoylphosphatidylcholine (DSPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, and combinations thereof.

### III. F. Constrictions

### III.F.1. Microfluidic Channels

As described herein, a constriction is used to cause a physical deformity in the cells, such that perturbations are created within the cell membrane of the cells, allowing for the delivery of a payload (e.g., gene-editing payload) into the cell. In some aspects, a constriction is within a channel contained within a microfluidic device (referred to herein as “microfluidic channel” or “channel”). Where multiple channels are involved, in some aspects, the multiple channels can be placed in parallel and/or in series within the microfluidic device. In some aspects, the cells described herein can be passed through at least about 2, at least about 3, at least about 4, at least about 5, at least about 6, at least about 7, at least about 8, at least about 9, at least about 10, at least about 20, at least about 30, at least about 40, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1,000 or more separate constrictions. In some aspects, the cells described herein are passed through more than about 1,000 separate constrictions. In some aspects, each of the constrictions are the same (e.g., has the same length, width, and/or depth). In some aspects, one or more of the constrictions are different.

Where plurality of constrictions are used, the plurality of constrictions can comprise a first constriction which is associated with a first payload (e.g., first gene-editing payload), and a second constriction which is associated with a second payload (e.g., second gene-editing payload), wherein the cell suspension passes through the first constriction such that the first payload is delivered to one or more cells of the plurality of cells, and then the cell suspension passes through the second constriction such that the second payload is delivered to the one or more cells of the plurality of cells. In some aspects, the cell suspension is passed through the second constriction immediately after the cell suspension passes through the first constriction (e.g., within less than about 1 second, e.g., within about 1 μs). In some aspects, after passing through the first constriction, a period of time elapses before the cell suspension is passed through the second constriction. In some aspects, the cell suspension is passed through the second constriction at least about 1 minute, at least about 30 minutes, at least about 1 hour, at least about 6 hours, at least about 12 hours, or at least about 1 day after the cell suspension is passed through the first constriction.

Exemplary microfluidic channels containing cell-deforming constrictions for use in the methods disclosed herein are described in US Publ. No. 2020/0277566 A1, US Publ. No. 2020/0332243 A1, US Publ. No. 2020/0316604 A1, U.S. Provisional Appl. No. 63/131,423, and U.S. Provisional Appl. No. 63/131,430, each of which is incorporated herein by reference in its entirety.

In some aspects, a microfluidic channel described herein (i.e., comprising a constriction) includes a lumen and is configured such that a cell suspended in a buffer (e.g., cell suspension) can pass through the channel. Microfluidic channels useful for the present disclosure can be made using any suitable materials available in the art, including, but not limited to, silicon, metal (e.g., stainless steel), plastic (e.g., polystyrene), ceramics, glass, crystalline substrates, amorphous substrates, polymers (e.g., Poly-methyl methacrylate (PMMA), PDMS, Cyclic Olefin Copolymer (COC)), or combinations thereof. In some aspects, the material is silicon. Fabrication of the microfluidic channel can be performed by any method known in the art, including, but not limited to, dry etching for example deep reactive ion etching, wet etching, photolithography, injection molding, laser ablation, SU-8 masks, or combinations thereof. In some aspects, the fabrication is performed using dry etching.

In some aspects, a microfluidic channel useful for the present disclosure comprises an entrance portion, a centerpoint, and an exit portion. In some aspects, the cross-section of one or more of the entrance portion, the centerpoint, and/or the exit portion can vary. For example, the cross-section can be circular, elliptical, an elongated slit, square, hexagonal, or triangular in shape.

The entrance portion defines a constriction angle. In some aspects, by modulating (e.g., increasing or decreasing) the constriction angle, any clogging of the constriction can be reduced or prevented. In some aspects, the angle of the exit portion can also be modulated. For example, in some aspects, the angle of the exit portion can be configured to reduce the likelihood of turbulence that can result in non-laminar flow. In some aspects, the walls of the entrance portion and/or the exit portion are linear. In some aspects, the walls of the entrance portion and/or the exit portion are curved.

In some aspects, the length, depth, and/or width of the constriction can vary. In some aspects, by modulating (e.g., increasing or decreasing) the length, depth, and/or width of the constriction, the delivery efficiency of a payload can be regulated.

In some aspects, the constriction has a length of less than 1 μm. In some aspects the constriction has a length of about 1 μm to about 100 μm. In some aspects, the constriction has a length of less than 1 μm, about 1 μm, about 5 μm, about 10 μm, about 20 μm, about 30 μm, about 40 μm, about 50 μm, about 60 μm, about 70 μm, about 80 μm, about 90 μm, or about 100 μm. In some aspects, the constriction has a length of about 10 μm. In some aspects, the constriction has a length of about 30 μm. In some aspects, the constriction has a length of about 70 μm. In some aspects, the constriction has a depth of about 5 μm to about 90 μm. In some aspects, the constriction has a depth greater than or equal to about 5 μm, about 10 μm, about 20 μm, about 30 μm, about 40 μm, about 50 μm, about 60 μm, about 70 μm, about 80 μm, about 90 μm, about 100 μm, about 110 μm, or about 120 μm. In some aspects, the constriction has a depth of about 10 μm. In some aspects, the constriction has a depth of about 20 μm. In some aspects, the constriction has a depth of about 70 μm. In some aspects, the constriction has a width of about 1 μm to about 10 μm (e.g., 3 μm to about 10 μm). In some aspects, the constriction has a width of about 1 μm, about 2 μm, about 3 μm, about 4 μm, about 4.5 μm, about 5 μm, about 6 μm, about 7 μm, about 8 μm, about 9 μm, or about 10 μm. In some aspects, the constriction has a width of about 6 μm. In some aspects, the constriction has a width of about 4 μm. In some aspects, the constriction has a width of about 3.5 μm. In some aspects, the constriction has a length of 10 μm, width of 6 μm, and a depth of 70 μm. In some aspects, the constriction has a length of 30 μm, width of 4 μm, and a depth of 70 μm. In some aspects, the constriction has a length of 30 μm, width of 3.5 μm, and a depth of 70 μm.

In some aspects, the diameter of a constriction (e.g., contained within a microfluidic channel) is a function of the diameter of one or more cells that are passed through the constriction. Not to be bound by any one theory, in some aspects, the diameter of the constriction is less than that of the cells, such that a deforming force is applied to the cells as they pass through the constriction, resulting in the transient physical deformity of the cells.

Accordingly, in some aspects, the diameter of the constriction (also referred to herein as “constriction size”) is about 20% to about 99% of the diameter of the cell. In some aspects, the constriction size is about 20%, about 30%, about 40%, about 50%, about 60%, about 70%, about 80%, about 90%, or about 99% of the cell diameter. As is apparent from the present disclosure, by modulating (e.g., increasing or decreasing) the diameter of a constriction, the delivery efficiency of a payload into a cell can also be regulated.

### III.F.2. Surfaces Having Pores

In some aspects, a constriction described herein comprises a pore, which is contained in a surface. Non-limiting examples of pores contained in a surface that can be used with the present disclosure are described in, e.g., US Publ. No. 2019/0382796 A1, which is incorporated herein by reference in its entirety.

In some aspects, a surface useful for the present disclosure (i.e., comprising one or more pores that can cause a physical deformity in a cell as it passes through the pore) can be made using any suitable materials available in the art and/or take any one of a number of forms. Non-limiting examples of such materials include synthetic or natural polymers, polycarbonate, silicon, glass, metal, alloy, cellulose nitrate, silver, cellulose acetate, nylon, polyester, polyethersulfone, Polyacrylonitrile (PAN), polypropylene, PVDF, polytetrafluorethylene, mixed cellulose ester, porcelain, ceramic, or combinations thereof.

In some aspects, the surface comprises a filter. In some aspects, the filter is a tangential flow filter. In some aspects, the surface comprises a membrane. In some aspects, the surface comprises a sponge or sponge-like matrix. In some aspects, the surface comprises a matrix. In some aspects, the surface comprises a tortuous path surface. In some aspects, the tortuous path surface comprises cellulose acetate.

The surface disclosed herein (i.e., comprising one or more pores) can have any suitable shape known in the art. Where the surface has a 2-dimensional shape, the surface can be, without limitation, circular, elliptical, round, square, star-shaped, triangular, polygonal, pentagonal, hexagonal, heptagonal, or octagonal. In some aspects, the surface is round in shape. Where the surface has a 3-dimensional shape, in some aspects, the surface can be, without limitation, cylindrical, conical, or cuboidal.

As is apparent from the present disclosure, a surface that is useful for the present disclosure (e.g., comprising one or more pores) can have various cross-sectional widths and thicknesses. In some aspects, the cross-sectional width of the surface is between about 1 mm and about 1 m. In some aspects, the surface has a defined thickness. In some aspects, the surface thickness is uniform. In some aspects, the surface thickness is variable. For example, in some aspects, certain portions of the surface are thicker or thinner than other portions of the surface. In such aspects, the thickness of the different portions of the surface can vary by about 1% to about 90%. In some aspects, the surface is between about 0.01 μm to about 5 mm in thickness.

The cross-sectional width of the pores can depend on the type of cell that is being targeted with a payload. In some aspects, the pore size is a function of the diameter of the cell of cluster of cells to be targeted. In some aspects, the pore size is such that a cell is perturbed (i.e., physically deformed) upon passing through the pore. In some aspects, the pore size is less than the diameter of the cell. In some aspects, the pore size is about 20% to about 99% of the diameter of the cell. In some aspects, the pore size is about 20%, about 30%, about 40%, about 50%, about 60%, about 70%, about 80%, about 90%, or about 99% of the diameter of the cell. In some aspects, the pore size is about 0.4 μm, about 0.5 μm, about 0.6 μm, about 0.7 μm, about 0.8 μm, about 0.9 μm, about 1 μm, about 2 μm, about 3 μm, about 4 μm, about 5 μm, about 6 μm, about 7 μm, about μm, about 9 μm, about 10 μm, about 11 μm, about 12 μm, about 13 μm, about 14 μm, or about 15 μm or more.

The entrances and exits of a pore can have a variety of angles. In some aspects, by modulating (e.g., increasing or decreasing) the pore angle, any clogging of the pore can be reduced or prevented. In some aspects, the flow rate (i.e., the rate at which a cell or a suspension comprising the cell passes through the pore) is between about 0.001 mL/cm/sec to about 100 L/cm/sec. For example, the angle of the entrance or exit portion can be between about 0 and about 90 degrees. In some aspects, the pores have identical entrance and exit angles. In some aspects, the pores have different entrance and exit angles. In some aspects, the pore edge is smooth, e.g., rounded or curved. As used herein, a “smooth” pore edge has a continuous, flat, and even surface without bumps, ridges, or uneven parts. In some aspects, the pore edge is sharp. As used herein, a “sharp” pore edge has a thin edge that is pointed or at an acute angle. In some aspects, the pore passage is straight. As used herein, a “straight” pore passage does not contain curves, bends, angles, or other irregularities. In some aspects, the pore passage is curved. As used herein, a “curved” pore passage is bent or deviates from a straight line. In some aspects, the pore passage has multiple curves, e.g. about 2, about 3, about 4, about 5, about 6, about 7, about 8, about 9, about 10 or more curves.

The pores can have any shape known in the art, including a 2-dimensional or 3-dimensional shape. The pore shape (e.g., the cross-sectional shape) can be, without limitation, circular, elliptical, round, square, star-shaped, triangular, polygonal, pentagonal, hexagonal, heptagonal, and octagonal. In some aspects, the cross-section of the pore is round in shape. In some aspects, the 3-dimensional shape of the pore is cylindrical or conical. In some aspects, the pore has a fluted entrance and exit shape. In some aspects, the pore shape is homogenous (i.e., consistent or regular) among pores within a given surface. In some aspects, the pore shape is heterogeneous (i.e., mixed or varied) among pores within a given surface.

A surface useful for the present disclosure can have a single pore. In some aspects, a surface useful for the present disclosure comprises multiple pores. In some aspects, the pores encompass about 10% to about 80% of the total surface area of the surface. In some aspects, the surface contains about 1.0×10⁵ to about 1.0×10³⁰ total pores. In some aspects, the surface comprises between about 10 and about 1.0×10¹⁵ pores per mm² surface area.

The pores can be distributed in numerous ways within a given surface. In some aspects, the pores are distributed in parallel within a given surface. In some aspects, the pores are distributed side-by-side in the same direction and are the same distance apart within a given surface. In some aspects, the distribution of the pores is ordered or homogeneous. In such aspects, the pores can be distributed in a regular, systematic pattern, or can be the same distance apart within a given surface. In some aspects, the distribution of the pores is random or heterogeneous. For instance, in some aspects, the pores are distributed in an irregular, disordered pattern, or are different distances apart within a given surface.

In some aspects, multiple surfaces are used, such that a cell passes through multiple pores, wherein the pores are on different surfaces. In some aspects, multiple surfaces are distributed in series. The multiple surfaces can be homogeneous or heterogeneous in surface size, shape, and/or roughness. The multiple surfaces can further contain pores with homogeneous or heterogeneous pore size, shape, and/or number, thereby enabling the simultaneous delivery of a range of payloads into different cell types.

In some aspects, an individual pore, e.g., of a surface that can be used with the present disclosure, has a uniform width dimension (i.e., constant width along the length of the pore passage). In some aspects, an individual pore has a variable width (i.e., increasing or decreasing width along the length of the pore passage). In some aspects, pores within a given surface have the same individual pore depths. In some aspects, pores within a given surface have different individual pore depths. In some aspects, the pores are immediately adjacent to each other. In some aspects, the pores are separated from each other by a distance. In some aspects, the pores are separated from each other by a distance of about 0.001 μm to about 30 mm.

In some aspects, the surface is coated with a material. The material can be selected from any material known in the art, including, without limitation, Teflon, an adhesive coating, surfactants, proteins, adhesion molecules, antibodies, anticoagulants, factors that modulate cellular function, nucleic acids, lipids, carbohydrates, transmembrane proteins, or combinations thereof. In some aspects, the surface is coated with polyvinylpyrrolidone. In some aspects, the material is covalently attached to the surface. In some aspects, the material is non-covalently attached to the surface. In some aspects, the surface molecules are released at the cells pass through the pores.

In some aspects, the surface has modified chemical properties. In some aspects, the surface is hydrophilic. In some aspects, the surface is hydrophobic. In some aspects, the surface is charged. In some aspects, the surface is positively and/or negatively charged. In some aspects, the surface can be positively charged in some regions and negatively charged in other regions. In some aspects, the surface has an overall positive or overall negative charge. In some aspects, the surface can be any one of smooth, electropolished, rough, or plasma treated. In some aspects, the surface comprises a zwitterion or dipolar compound. In some aspects, the surface is plasma treated.

In some aspects, the surface is contained within a larger module. In some aspects, the surface is contained within a syringe, such as a plastic or glass syringe. In some aspects, the surface is contained within a plastic filter holder. In some aspects, the surface is contained within a pipette tip.

### III.G. Cell Perturbation

As described herein, as a cell passes through a constriction, it becomes physically deformed, such that there is a perturbation (e.g., a hole, tear, cavity, aperture, pore, break, gap, perforation) in the cell membrane of the cell. Such perturbation in the cell membrane is temporary and sufficient for any of the payloads (e.g., gene-editing payload) described herein to be delivered into the cell. Cells have self-repair mechanisms that allow the cells to repair any disruption in their cell membrane. See Blazek et al., Physiology (Bethesda) 30(6): 438-48 (November 2015), which is incorporated herein by reference in its entirety. Accordingly, in some aspects, once the cells have passed through the constriction (e.g., microfluidic channel or pores), the perturbations in the cell membrane can be reduced or eliminated, such that the payload that was delivered into the cell does not exit the cell.

In some aspects, the perturbation in the cell membrane lasts from about 1.0×10⁻⁹ seconds to about 2 hours after the pressure is removed (e.g., cells have passed through the constriction). In some aspects, the cell perturbation lasts for about 1.0×10⁻⁹ second to about 1 second, for about 1 second to about 1 minute, or for about 1 minute to about 1 hour. In some aspects, the cell perturbation lasts for between about 1.0×10⁻⁹ to about 1.0×10⁻¹, between about 1.0×10⁻⁹ to about 1.0×10⁻², between about 1.0×10⁻⁹ to about 1.0×10⁻³, between about 1.0×10⁻⁹ to about 1.0×10⁻⁴, between about 1.0×10⁻⁹ to about 1.0×10⁻⁵, between about 1.0×10⁻⁹ to about 1.0×10⁻⁶, between about 1.0×10⁻⁹ to about 1.0×10⁻⁷, or between about 1.0×10⁻⁹ to about 1.0×10⁻⁸ seconds. In some aspects, the cell perturbation lasts for about 1.0×10⁻⁸ to about 1.0×10⁻¹, for about 1.0×10⁻⁷ to about 1.0×10⁻¹, about 1.0×10⁻⁶ to about 1.0×10⁻¹, about 1.0×10⁻⁵ to about 1.0×10⁻¹, about 1.0×10⁻⁴ to about 1.0×10⁻¹, about 1.0×10⁻³ to about 1.0×10⁻¹, or about 1.0×10⁻² to about 1.0×10⁻¹ seconds. The cell perturbations (e.g., pores or holes) created by the methods described herein are not formed as a result of assembly of polypeptide subunits to form a multimeric pore structure such as that created by complement or bacterial hemolysins.

In some aspects, as the cell passes through the constriction, the pressure applied to the cells temporarily imparts injury to the cell membrane that causes passive diffusion of material through the perturbation. In some aspects, the cell is only deformed or perturbed for a brief period of time, e.g., on the order of 100 us or less to minimize the chance of activating apoptotic pathways through cell signaling mechanisms, although other durations are possible (e.g., ranging from nanoseconds to hours). In some aspects, the cell is deformed for less than about 1.0×10⁻⁹ seconds to less than about 2 hours. In some aspects, the cell is deformed for less than about 1.0×10⁻⁹ second to less than about 1 second, less than about 1 second to less than about 1 minute, or less than about 1 minute to less than about 1 hour. In some aspects, the cell is deformed for about 1.0×10⁻⁹ seconds to about 2 hours. In some aspects, the cell is deformed for about 1.0×10⁻⁹ second to about 1 second, about 1 second to about 1 minute, or about 1 minute to about 1 hour. In some aspects, the cell is deformed for between any one of about 1.0×10⁻⁹ seconds to about 1.0×10⁻¹ seconds, about 1.0×10⁻⁹ seconds to about 1.0×10⁻² seconds, about 1.0×10⁻⁹ seconds to about 1.0×10⁻³ seconds, about 1.0×10⁻⁹ seconds to about 1.0×10⁻⁴ seconds, about 1.0×10⁻⁹ seconds to about 1.0×10⁻⁵ seconds, about 1.0×10⁻⁹ seconds to about 1.0×10⁻⁶ seconds, about 1.0×10⁻⁹ seconds to about 1.0×10⁻⁷ seconds, or about 1.0×10⁻⁹ seconds to about 1.0×10⁻⁸ seconds. In some aspects, the cell is deformed or perturbed for about 1.0×10⁻⁸ seconds to about 1.0×10⁻¹ seconds, for about 1.0×10⁻⁷ seconds to about 1.0×10⁻¹ seconds, about 1.0×10⁻⁶ seconds to about 1.0×10⁻¹ seconds, about 1.0×10⁻⁵ seconds to about 1.0×10⁻¹ seconds, about 1.0×10⁻⁴ seconds to about 1.0×10⁻¹ seconds, about 1.0×10⁻³ seconds to about 1.0×10⁻¹ seconds, or about 1.0×10⁻² seconds to about 1.0×10⁻¹ seconds. In some aspects, deforming the cell includes deforming the cell for a time ranging from, without limitation, about 1 us to at least about 750 μs, e.g., at least about 1 μs, at least about 10 μs, at least about 50 μs, at least about 100 μs, at least about 500 μs, or at least about 750 μs.

In some aspects, the delivery of a payload (e.g., gene-editing payload) into the cell occurs simultaneously with the cell passing through the constriction. In some aspects, delivery of the payload into the cell can occur after the cell passes through the constriction (i.e., when perturbation of the cell membrane is still present and prior to cell membrane of the cells being restored). In some aspects, delivery of the payload into the cell occurs on the order of minutes after the cell passes through the constriction. In some aspects, a perturbation in the cell after it passes through the constriction is corrected within the order of about five minutes after the cell passes through the constriction.

In some aspects, the viability of a cell (e.g., stem cell or PBMC) after passing through a constriction is about 5% to about 100%. In some aspects, the cell viability after passing through the constriction is at least about 5%, at least about 10%, at least about 20%, at least about 30%, at least about 40%, at least about 50%, at least about 60%, at least about 70%, at least about 75%, at least about 80%, at least about 85%, at least about 90%, at least about 95%, or at least about 99%. In some aspects, the cell viability can be measured from about 1.0×10⁻² seconds to at least about 10 days after the cell passes through the constriction. For example, the cell viability can be measured from about 1.0×10⁻² seconds to about 1 second, about 1 second to about 1 minute, about 1 minute to about 30 minutes, or about 30 minutes to about 2 hours after the cell passes through the constriction. In some aspects, the cell viability can be measured about 1.0×10⁻² seconds to about 2 hours, about 1.0×10⁻² seconds to about 1 hour, about 1.0×10⁻² seconds to about 30 minutes, about 11.0×10⁻² seconds to about 1 minute, about 1.0×10⁻² seconds to about 30 seconds, about 1.0×10⁻² seconds to about 1 second, or about 1.0×10⁻² seconds to about 0.1 second after the cell passes through the constriction. In some aspects, the cell viability is measured about 1.5 hours to about 2 hours, about 1 hour to about 2 hours, about 30 minutes to about 2 hours, about 15 minutes to about 2 hours, about 1 minute to about 2 hours, about 30 seconds to about 2 hours, or about 1 second to about 2 hours after the cell passes through the constriction. In some aspects, the cell viability is measured about 2 hours to about 5 hours, about 5 hours to about 12 hours, about 12 hours to about 24 hours, or about 24 hours to about 10 days after the cell passes through the constriction.

### III.H. Delivery Parameters

As is apparent from the present disclosure, a number of parameters can influence the delivery efficiency of a payload (e.g., gene-editing payload) into a cell using the squeeze processing methods provided herein. Accordingly, by modulating (e.g., increasing or decreasing) one or more of the delivery parameters, the delivery of a payload into a cell can be improved. Therefore, in some aspects, the present disclosure relates to a method of increasing the delivery of a payload (e.g., gene-editing payload) into a cell, wherein the method comprises modulating one or more parameters under which a cell suspension is passed through a constriction, wherein the cell suspension comprises a population of the cells, and wherein the one or more parameters increase the delivery of a payload into one or more cells of the population of cells compared to a reference parameter. As described else wherein the present disclosure, the payload can be in contact with the population of cells before, during, or after the squeezing step.

In some aspects, by modulating one or more of the delivery parameters, the delivery of the payload (e.g., gene-editing payload) into the one or more cells is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, compared to a delivery of the payload into a corresponding cell using the reference parameter.

In some aspects, the one or more delivery parameters that can be modulated to increase the delivery efficiency of a parameter comprises a cell density (i.e., the concentration of the cells present, e.g., in the cell suspension), pressure, or both. Additional examples of delivery parameters that can be modulated are provided elsewhere in the present disclosure.

In some aspects, the cell density is about 1×10³ cells/mL, about 1×10⁴ cells/mL, about 1×10⁵ cells/mL, about 1×10⁶ cells/mL, about 2×10⁶ cells/mL, about 3×10⁶ cells/mL, about 4×10⁶ cells/mL, about 5×10⁶ cells/mL, about 6×10⁶ cells/mL, about 7×10⁶ cells/mL, about 8×10⁶ cells/mL, about 9×10⁶ cells/mL, about 1×10⁷ cells/mL, about 2×10⁷ cells/mL, about 3×10⁷ cells/mL, about 4×10⁷ cells/mL, about 5×10⁷ cells/mL, about 6×10⁷ cells/mL, about 7×10⁷ cells/mL, about 8×10⁷ cells/mL, about 9×10⁷ cells/mL, about 1×10⁸ cells/mL, about 1.1×10⁸ cells/mL, about 1.2×10⁸ cells/mL, about 1.3× 10⁸ cells/mL, about 1.4×10⁸ cells/mL, about 1.5×10⁸ cells/mL, about 2.0×10⁸ cells/mL, about 3.0×10⁸ cells/mL, about 4.0×10⁸ cells/mL, about 5.0×10⁸ cells/mL, about 6.0×10⁸ cells/mL, about 7.0×10⁸ cells/mL, about 8.0×10⁸ cells/mL, about 9.0×10⁸ cells/mL, about 1.0×10⁹ cells/mL, about 2.0×10⁹ cells/mL, about 3.0×10⁹ cells/mL, about 4×10⁹ cells/mL, or about 5×10⁹ cells/mL or more. In some aspects, the cell density is between about 6×10⁷ cells/mL and about 1.2×10⁸ cells/mL.

In some aspects, the pressure is about 1 psi, about 2 psi, about 3 psi, about 4 psi, about 5 psi, about 6 psi, about 7 psi, about 8 psi, about 9 psi, about 10 psi, about 15 psi, about 20 psi, about 25 psi, about 30 psi, about 35 psi, about 40 psi, about 50 psi, about 55 psi, about 60 psi, about 65 psi, about 70 psi, about 75 psi, about 80 psi, about 85 psi, about 90 psi, about 95 psi, about 100 psi, about 105 psi, about 110 psi, about 120 psi, about 130 psi, about 140 psi, about 150 psi, about 160 psi, about 170 psi, about 180 psi, about 190 psi, or about 200 psi or more. In some aspects, the pressure is between about 30 psi and about 110 psi. In some aspects, the pressure is about 105 psi.

In some aspects, the particular type of device (e.g., microfluidic chip) can also have an effect on the delivery efficiency of a payload described herein (e.g., gene-editing payload). In the case of a microfluidic chip, different chips can have different constriction parameters, e.g., length, depth, and width of the constriction; entrance angle, exit angle, length, depth, and width of the approach region, etc. As described herein, such variables can influence the delivery of a payload into a cell using the squeeze processing methods of the present disclosure. In some aspects, the length of the constriction is up to 100 μm. For instance, in some aspects, the length is about 1 μm, about 5 μm, 10 μm, about 20 μm, about 30 μm, about 40 μm, about 50 μm, about 60 μm, about 70 μm, about 80 μm, about 90 μm, or about 100 μm. In some aspects, the length of the constriction is less than 1 μm. In some aspects, the length of the constriction is less than about 1 μm, less than about 5 μm, less than about 10 μm, less than about 20 μm, less than about 30 μm, less than about 40 μm, less than about 50 μm, less than about 60 μm, less than about 70 μm, less than about 80 μm, less than about 90 μm, or less than about 100 μm. In some aspects, the constriction has a length of about 10 μm. In some aspects, the constriction has a length of about 30 μm. In some aspects, the constriction has a length of about 70 μm.

In some aspects, the width of the constriction is up to about 10 μm. In some aspects, the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm. In some aspects, the width is between about 3 μm to about 10 μm. In some aspects, the width is about 3 μm, about 4 μm, about 5 μm, about 6 μm, about 7 μm, about 8 μm, about 9 μm, or about 10 μm. In some aspects, the width of the constriction is about 6 μm. In some aspects, the width of the constriction is about 4 μm. In some aspects, the width of the constriction is about 3.5 μm.

In some aspects, the depth of the constriction is at least about 1 μm. In some aspects, the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm. In some aspects, the depth is between about 5 μm to about 90 μm. In some aspects, the depth is about 5 μm, about 10 μm, about 15 μm, about 20 μm, about 30 μm, about 40 μm, about 50 μm, about 60 μm, about 70 μm, about 80 μm, or about 90 μm. In some aspects, the depth of the constriction is about 70 μm. In some aspects, the depth of the constriction is about 20 μm.

In some aspects, the length is about 10 μm, the width is about 6 μm, and depth is about 70 μm. In some aspects, the constriction has a length of 30 μm, width of 4 μm, and a depth of 70 μm. In some aspects, the constriction has a length of 30 μm, width of 3.5 μm, and a depth of 70 μm.

Additional examples of parameters that can influence the delivery of a payload into the cell include, but are not limited to, the dimensions of the constriction (e.g., length, width, and/or depth), the entrance angle of the constriction, the surface properties of the constrictions (e.g., roughness, chemical modification, hydrophilic, hydrophobic), the operating flow speeds, payload concentration, the amount of time that the cell recovers, or combinations thereof. Further parameters that can influence the delivery efficiency of a payload (e.g., gene-editing payload) can include the velocity of the cell in the constriction, the shear rate in the constriction, the viscosity of the cell suspension, the velocity component that is perpendicular to flow velocity, and time in the constriction. Such parameters can be designed to control delivery of the payload.

In some aspects, the temperature used in the methods of the present disclosure can also have an effect on the delivery efficiency of the payloads into the cell, as well as the viability of the cell. In some aspects, the squeeze processing method is performed between about −5° C. and about 45° C. For example, the methods can be carried out at room temperature (e.g., about 20° ° C.), physiological temperature (e.g., about 37° C.), higher than physiological temperature (e.g., greater than about 37° C. to 45° C. or more), or reduced temperature (e.g., about −5° ° C. to about 4° C.), or temperatures between these exemplary temperatures.

Various methods can be utilized to drive the cells through the constrictions. For example, pressure can be applied by a pump on the entrance side (e.g., gas cylinder, or compressor), a vacuum can be applied by a vacuum pump on the exit side, capillary action can be applied through a tube, and/or the system can be gravity fed. Displacement based flow systems can also be used (e.g., syringe pump, peristaltic pump, manual syringe or pipette, pistons, etc.). In some aspects, the cells are passed through the constrictions by positive pressure. In some aspects, the cells are passed through the constrictions by constant pressure or variable pressure. In some aspects, pressure is applied using a syringe. In some aspects, pressure is applied using a pump. In some aspects, the pump is a peristaltic pump or a diaphragm pump. In some aspects, pressure is applied using a vacuum. In some aspects, the cells are passed through the constrictions by g-force. In some aspects, the cells are passed through the constrictions by capillary pressure.

In some aspects, fluid flow directs the cells through the constrictions. In some aspects, the fluid flow is turbulent flow prior to the cells passing through the constriction. Turbulent flow is a fluid flow in which the velocity at a given point varies erratically in magnitude and direction. In some aspects, the fluid flow through the constriction is laminar flow. Laminar flow involves uninterrupted flow in a fluid near a solid boundary in which the direction of flow at every point remains constant. In some aspects, the fluid flow is turbulent flow after the cells pass through the constriction. The velocity at which the cells pass through the constrictions can be varied. In some aspects, the cells pass through the constrictions at a uniform cell speed. In some aspects, the cells pass through the constrictions at a fluctuating cell speed.

In some aspects, a combination treatment is used to deliver a payload, e.g., the methods described herein followed by exposure to an electric field downstream of the constriction. In some aspects, the cell is passed through an electric field generated by at least one electrode after passing through the constriction. In some aspects, the electric field assists in delivery of a payload to a second location inside the cell such as the cell nucleus. In some aspects, one or more electrodes are in proximity to the cell-deforming constriction to generate an electric field. In some aspects, the electric field is between about 0.1 kV/m to about 100 MV/m. In some aspects, an integrated circuit is used to provide an electrical signal to drive the electrodes. In some aspects, the cells are exposed to the electric field for a pulse width of between about 1 ns to about 1 s and a period of between about 100 ns to about 10 s.

### III.I. Therapeutic Uses

In some aspects, the present disclosure relates to the use of the cells produced using the squeeze processing methods described herein to treat various diseases or disorders. As is apparent from the present disclosure, the methods and compositions provided herein can be useful for diseases and disorders where cell-based therapies (e.g., cell replacement therapy or adoptive cell therapy) can be used as a treatment. By replacing cells that are damaged with the cells produced using the methods provided herein (e.g., genetically modified to express an increased or reduced expression of a gene), in some aspects, one or more functions associated with the damaged cells can be restored, and thereby, treat the disease or disorder. Alternatively, cells (e.g., T cells) can be modified to modulate gene expression, such that the cells can, e.g., exhibit improved therapeutic effects or express proteins that the cells would not normally express (e.g., chimeric antigen receptor).

### IV. Compositions of the Disclosures

In some aspects, the disclosure provides a system for delivery of a payload (e.g., gene-editing payload) into a cell, the system comprising a microfluidic channel described herein, a cell suspension comprising a plurality of the cells and the payload; wherein the constriction is configured such that the plurality of cells can pass through the microfluidic channel, wherein the passing of the plurality of cells causes a deformity and disruption of the cell membrane of the cell, allowing the payload to enter the cell.

In some aspects, the disclosure provides a system for delivering a payload, the system comprising a surface with pores, a cell suspension comprising a plurality of the cells and the payload; wherein the surface with pores is configured such that the plurality of cells can pass through the pores, wherein the passing of the plurality of cells causes a deformity and disruption of the cell membrane of the cell, allowing the payload to enter the cell. In some aspects, the surface is a filter or a membrane. In some aspects of the above aspects, the system further comprises at least one electrode to generate an electric field. In some aspects, the system is used to deliver a payload into a cell by any of the methods described herein. The system can include any aspect described for the methods disclosed above, including microfluidic channels or a surface having pores to provide cell-deforming constrictions, cell suspensions, cell perturbations, delivery parameters. In some aspects, the delivery parameters, such as operating flow speeds, cell and compound concentration, velocity of the cell in the constriction, and the composition of the cell suspension (e.g., osmolarity, salt concentration, serum content, cell concentration, pH, etc.) are optimized for delivery of a payload (e.g., gene-editing payload) into the cell.

In some aspects, the disclosure provides a cell produced using any of the methods provided herein (e.g., modified T cells with increased or reduced expression of a gene). In some aspects, provided herein is a cell comprising a perturbation in the cell membrane, wherein the perturbation is due to one or more parameters which deform the cell (e.g., delivery parameters described herein), thereby creating the perturbation in the cell membrane of the cell such that a payload (e.g., gene-editing payload) can enter the cell. In some aspects, provided herein is a cell comprising a payload (e.g., gene-editing payload), wherein the payload entered the cell through a perturbation in the cell membrane, which was due to one or more parameters which deform the cell (e.g., delivery parameters described herein) and thereby creating the perturbation in the cell membrane of the cell such that the payload entered the cell. In some aspects, such cells can comprise any of the cells described herein (e.g., stem cells or PBMCs).

In some aspects, the present disclosure provides a composition comprising a plurality of cells, wherein the plurality of cells were produced by any of the methods provided herein. Also provided herein is a composition comprising a population of cells and a payload (e.g., gene-editing payload) under one or more parameters, which result in deformation of one or more cells of the population of cells and thereby creating perturbations in the cell membrane of the one or more cells, and wherein the perturbations in the cell membrane allows the payload to enter the one or more cells.

Also provided are kits or articles of manufacture for use in delivering into a cell a payload (e.g., gene-editing payload) as described herein. In some aspects, the kits comprise the compositions described herein (e.g. a microfluidic channel or surface containing pores, cell suspensions, and/or payload) in suitable packaging. Suitable packaging materials are known in the art, and include, for example, vials (such as sealed vials), vessels, ampules, bottles, jars, flexible packaging (e.g., sealed Mylar or plastic bags), and the like. These articles of manufacture can further be sterilized and/or sealed.

The present disclosure also provides kits comprising components of the methods described herein and can further comprise instruction(s) for performing said methods to deliver a payload (e.g., gene-editing payload) into a cell. The kits described herein can further include other materials, including other buffers, diluents, filters, needles, syringes, and package inserts with instructions for performing any methods described herein; e.g., instructions for delivering a payload into a cell.

The following examples are offered by way of illustration and not by way of limitation.

### Examples

### Example 1: Co-Delivery of Multiple Payloads Using Squeeze Processing

To assess the ability of the delivery methods described herein to deliver multiple payloads (i.e., multiplex), mRNA and CRISPR/Cas9 ribonucleprotein (RNP) were co-delivered to unstimulated human T cells using squeeze processing, and then gene expression was assessed. Briefly, cryopreserved human PBMCs (Brigham and Women's Hospital, Boston, MA) were thawed, and T cells were isolated using a bead-based negative isolation kit (STEMCELL, Vancouver, CA). Cells were rested at 2×10⁶ cells/mL for 30 minutes in complete media with 100 U/mL IL-2 cytokine support. Cas9 RNPs specific to the beta-2 microglobulin (B2M) gene were pre-complexed using Cas9 protein (Aldevron, Fargo, ND) and in-house designed guides (Integrated DNA Technologies, Coralville, IA) at a 2.5:1 molar ratio of guide:Cas9 and allowed to complex at room temperature for 10 minutes, then stored on ice prior to use.

T cells were prepared at a final concentration of 20M/ml in X-VIVO™ 10 (Lonza, Basel, Switzerland) with 55 uM BME (Gibco, Waltham, MA), 100 ug/ml RNP, 100 ug/ml CD86 mRNA (Trilink, San Diego, CA), and 100 ug/ml 3 kDa Cascade Blue dextran (Thermo, Waltham, MA). The solution of cells with the delivery material was squeezed on a chip with 30 μm length, 3.5 μm width and 70 μm depth constriction at 105 psi, and immediately quenched in complete media, spun to wash, and resuspended at 2M/ml in complete media for a culture at 37° C. After two days, cells were stained with Live/Dead Near-infrared (Thermo) for viability, anti-B2M-APC (BioLegend, San Diego, CA) and anti-CD86-PE (BioLegend), and fluorescence was measured using flow cytometry (Thermo).

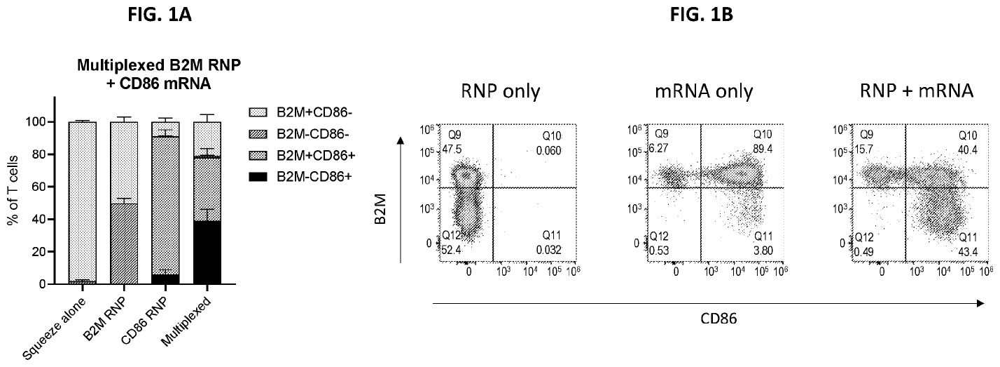

As shown in FIGS. 1A and 1B, in cells that were squeezed through the constriction with B2M RNP alone, there was approximately a 50% reduction in B2M surface expression (see B2M RNP group). Similarly, in cells that were squeezed through the constriction with CD86 mRNA alone, nearly 90% of the T cells were positive for CD86 expression. In cells that were squeezed through the constriction with both the B2M RNP and CD86 mRNA, all of the B2M-edited cells were also expressing CD86 (approximately 40% of the population); and, approximately half of the CD86+ cells were negative for B2M, consistent with the 50% editing efficiency.

These results demonstrate that squeeze processing delivery methods described herein can effectively co-deliver multiple payloads, such as two different types of gene editing tools, e.g., CRISPR/Cas9 RNPs and mRNA, with no loss of efficiency and expected levels of combined edits on a per-cell basis.

### Example 2: Multiplex Editing in Human T Cells Through Co-Delivery of Multiple RNPs Using Squeeze Processing

Next, it was determined whether multiple edits on a per cell basis can be achieved by co-delivering CRISPR/Cas9 RNPs to activated human T cells. Briefly, cryopreserved human PBMCs (Brigham and Women's Hospital, Boston, MA) were thawed and T cells were isolated using a bead-based negative isolation kit (STEMCELL, Vancouver, CA). Cells were rested overnight at 2M/ml in complete media with no cytokines at 37° C. The next day, cells were activated using anti-CD3/anti-CD28 Dynabeads per manufacturer's protocol (Thermo, Waltham, MA) and seeded at 1M cells/ml in complete media supplemented with 200 U IL-2/ml at 37° C. After 2 days of culture on beads, dead cells and beads were removed using a dead cell removal kit with magnet (STEMCELL). Cas9 ribonucleoproteins (RNPs) specific to the TRAC gene, B2M gene, or the TIM-3 gene were pre-complexed separately using Cas9 protein (Aldevron, Fargo, ND) and in-house designed guides (Integrated DNA Technologies, Coralville, IA) at a 2.5:1 molar ratio of guide:Cas9 and allowed to complex at room temperature for 10 minutes, then stored on ice prior to use. T cells were prepared at a final concentration of 20M/ml in X-VIVO™ 10 (Lonza, Basel, Switzerland) with 55 uM BME (Gibco, Waltham, MA), 100 ug/ml 3 kDa Cascade Blue dextran (Thermo), and 100 ug/ml of RNP. For multiplexed samples, all three RNPs were pre-complexed separately and then combined with cells at a concentration of 100 ug/ml each, and for individually edited samples, only one RNP was added to a final concentration of 100 ug/ml.

The solution of cells with delivery material (i.e., either all three RNPs in combination or separately) was squeezed on a chip with 30 μm length, 4 μm width and 70 μm depth constriction at 30 psi, immediately quenched in complete media, spun to wash, and resuspended at 2M/ml in complete media with 200 U IL-2/ml for culture at 37° C. After two days, cells were stained with Live/Dead Near-infrared (Thermo) for viability, anti-B2M-FITC (BioLegend, San Diego, CA), anti-TRAC(TCRa/b)-APC (BioLegend), and anti-CD3-PE (Biolegend), and fluorescence was measured using flow cytometry (Thermo).

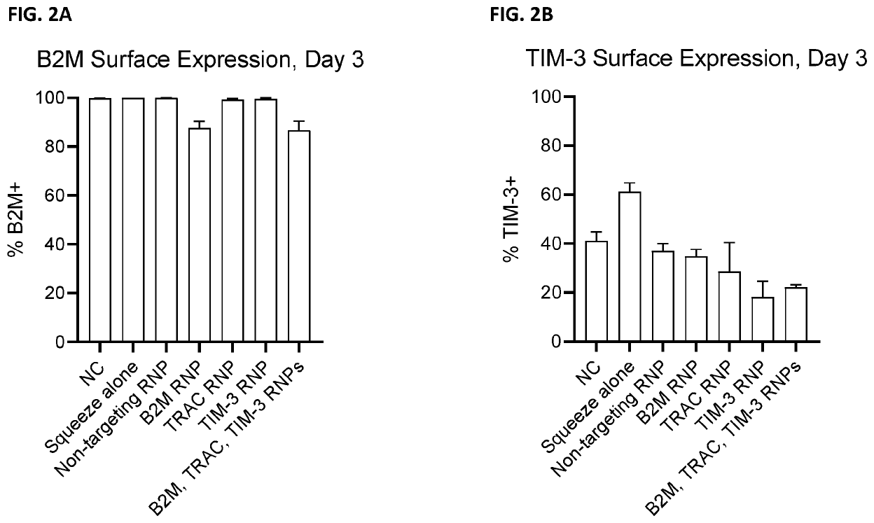

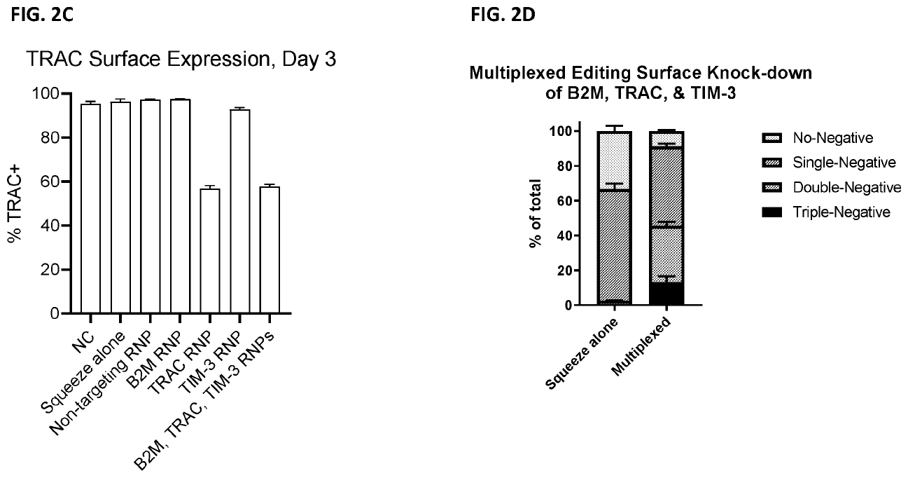

As shown in FIGS. 2A-2C, editing efficiency in a multiplexed, co-delivered setting was not different from single-RNP editing efficiencies. The multiple knock out efficiency was approximately 10% by surface staining and noting triple negative for all surface markers. FIG. 2D. These results demonstrate that the squeeze processing methods described herein can effectively co-deliver multiple RNPs to achieve a multi-edited cell population.

### Example 3: Sequential Delivery of Multiple RNPs Using Squeeze Processing to Achieve Editing of Multiple Targets in Human T Cells

Next, it was determined whether multiple edits on a per cell basis can be achieved by sequentially delivering CRISPR/Cas9 RNPs to activated human T cells. Briefly, cryopreserved human PBMCs (Brigham and Women's Hospital, Boston, MA) were thawed and T cells were isolated using a bead-based negative isolation kit (STEMCELL, Vancouver, CA). Cells were rested overnight at 2M/ml in complete media with no cytokines at 37° C. The next day, cells were activated using anti-CD3/anti-CD28 Dynabeads per manufacturer's protocol (Thermo, Waltham, MA) and seeded at 1M cells/ml in complete media supplemented with 200 U IL-2/ml at 37° C. After 2 days of culture post-bead activation, dead cells and beads were removed using a dead cell removal kit with magnet (STEMCELL). Cas9 ribonucleoproteins (RNPs) specific to the TRAC gene, B2M gene, or the TIM-3 gene were pre-complexed separately using Cas9 protein (Aldevron, Fargo, ND) and in-house designed guides (Integrated DNA Technologies, Coralville, IA) at a 2.5:1 molar ratio of guide:Cas9 and allowed to complex at room temperature for 10 minutes, then stored on ice prior to use. T cells were prepared at a final concentration of 20M/ml in X-VIVO™ 10 (Lonza, Basel, Switzerland) with 55 uM BME (Gibco, Waltham, MA), 100 ug/ml 3 kDa Cascade Blue dextran (Thermo), and 100 ug/ml of RNP.

The solution of cells with delivery material (i.e., TRAC-specific RNPs) was squeezed on a chip with 30 μm length, 4 μm width and 70 μm depth constriction at 30 psi, immediately quenched in complete media, spun to wash, and resuspended at 2M/ml in complete media with 200 U IL-2/ml for culture at 37° C. After two days, the cells were prepared for an additional squeeze processing (using the same parameters and conditions as the first squeeze processing) but with TIM-3-specific RNPs. The next day, those cells were prepared again for a third squeeze processing, in which B2M-specific RNPs were delivered to the cells (using the same parameters and conditions as the earlier two squeeze processings). Three days following the last RNP squeeze, cells were stained with Live/Dead Near-infrared (Thermo) for viability, anti-B2M-FITC (BioLegend, San Diego, CA), anti-TRAC(TCRa/b)-APC (BioLegend), and anti-CD3-PE (Biolegend), and fluorescence was measured using flow cytometry (Thermo).

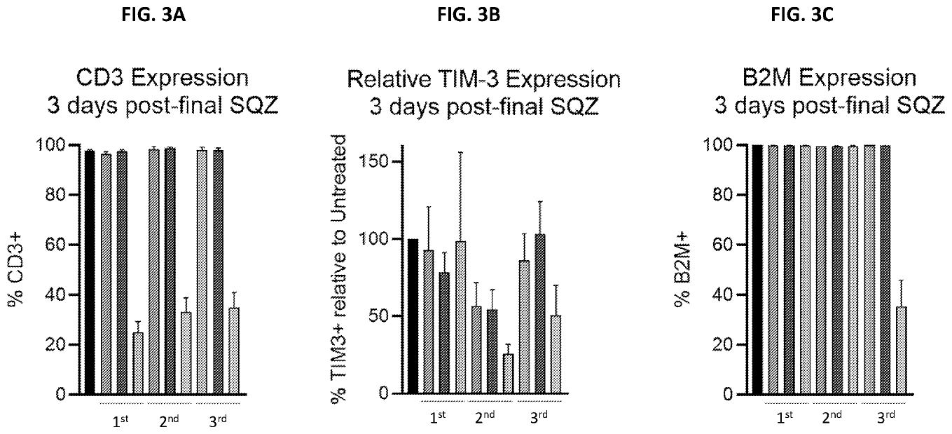

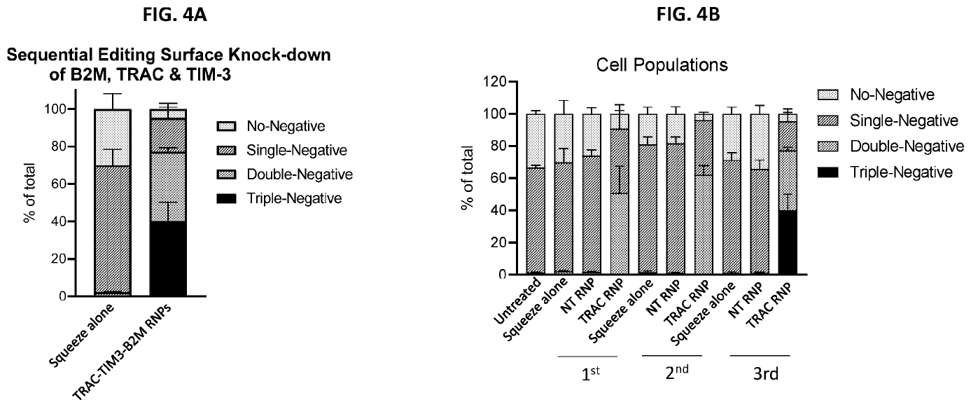

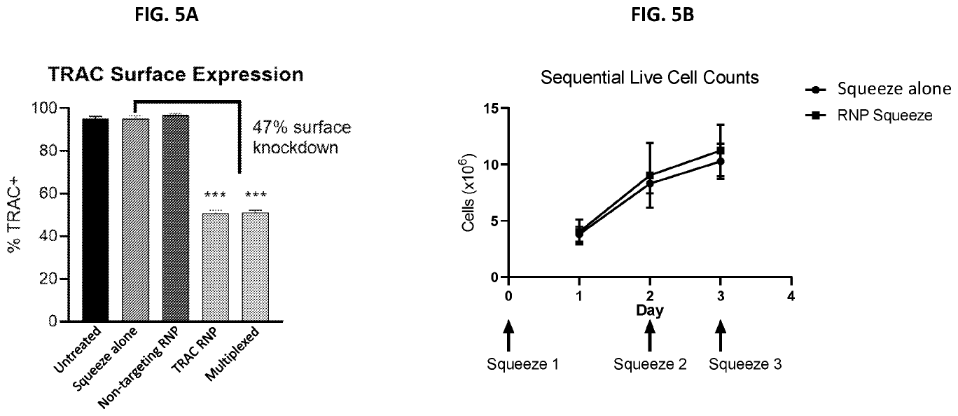

As shown in FIGS. 3A-3C, editing efficiencies of >50% were achieved for all three target genes (i.e., CD3, TIM-3, and B2M). Additionally, the prior editing effects were maintained with each subsequent squeeze processing (see, e.g., FIGS. 3A and 3B). In cells that sequentially received all three RNPs, an efficiency of 40% triple-negative cells was achieved (FIG. 4A). Prior to the sequential squeeze processing, there were very few TIM-3+ T cells. Therefore, although approximately 50% editing of TIM-3 was achieved, on multi-edited cells, there was negligible effect. When considering the multi-knock out of TRAC and B2M, two markers which express at near 100% pre-editing, there was again a 40% double-negative efficiency. And, lastly, as shown in FIG. 5B, delivery of the RNPs using sequential squeeze processing had minimal effect on the viability of the cells.

These results suggest a high degree of overlap in co-delivered cells which can be edited for multiple surface markers using the sequential method of RNP delivery. The results also demonstrate the safety of the sequential squeeze processing methods described herein.

### Example 4: Sequential Delivery of Multiple RNPs Using Squeeze Processing Results in Greater Multiple Editing Efficiency Compared to Co-Delivery

To further assess the delivery methods described herein, the editing efficiency of sequentially delivered CRISPR/Cas9 RNPs to co-delivery was compared. Briefly, cryopreserved human PBMCs (Brigham and Women's Hospital, Boston, MA) were thawed and T cells were isolated using a bead-based negative isolation kit (STEMCELL, Vancouver, CA). Cells were rested overnight at 2×10⁶ cells/ml in complete media with no cytokines at 37° C. The next day, cells were activated using anti-CD3/anti-CD28 Dynabeads per manufacturer's protocol (Thermo, Waltham, MA) and seeded at 1×10⁶ cells/ml in complete media supplemented with 200 U IL-2/ml at 37° C. After 2 days of culture post-bead activation, dead cells and beads were removed using a dead cell removal kit with magnet (STEMCELL). Cas9 ribonucleoproteins (RNPs) specific to the B2M, TRAC, or TIM-3 genes were pre-complexed separately using Cas9 protein (Aldevron, Fargo, ND) and in-house designed guides (Integrated DNA Technologies, Coralville, IA) at a 2.5:1 molar ratio of guide:Cas9 and allowed to complex at room temperature for 10 minutes, then stored on ice prior to use. T cells were prepared at a final concentration of 20×10⁶ cells/ml in X-VIVO™ 10 (Lonza, Basel, Switzerland) with 55 uM BME (Gibco, Waltham, MA), 100 ug/ml 3 kDa Cascade Blue dextran (Thermo), and 100 ug/ml of RNP. For individually edited samples, only one RNP was added to a final concentration of 100 ug/ml.

The solution of cells with delivery material was squeezed on a chip with 30 μm length, 4 μm width and 70 μm depth constriction at 30 psi, and immediately quenched in complete media, spun to wash, and resuspended at 2×10⁶ cells/ml in complete media for a culture at 37° C.

For multiplexed samples, the cells were squeezed with no cargo on the first day. Two days later, all three RNPs were pre-complexed separately and then combined with cells at a concentration of 100 ug/ml each and squeezed. For sequential editing, some of the sample which had received TRAC RNP on the first day was prepared for Cell Squeeze® as described and delivered with RNP for TIM-3 two days after the first squeeze. The next day, those cells were squeezed with RNP for B2M. Cells were analyzed for editing at least three days after the last RNP squeeze by staining with Live/Dead Near-infrared (Thermo) for viability, anti-B2M-FITC (BioLegend, San Diego, CA), anti-TRAC(TCRa/b)-APC (BioLegend), and anti-CD3-PE (Biolegend), and fluorescence was measured using flow cytometry (Thermo). Cells were collected for genomic DNA isolation (Qiagen, Hilden, Germany), and amplicons surrounding each edit location were amplified using PCR. These amplicons were submitted to CRISPR short amplicon deep sequencing. The results were analyzed using TIDE and ICE analyses. Cells were also collected for 10× genomics (Pleasanton, CA) single-cell analysis; 133,000 cells were taken from culture on Day 5, cells were centrifuged and resuspended in PBS+0.04% BSA, washed once, and transferred to the Whitehead institute for analysis of 100,000 cells using the 5′ RNA-Seq kit.

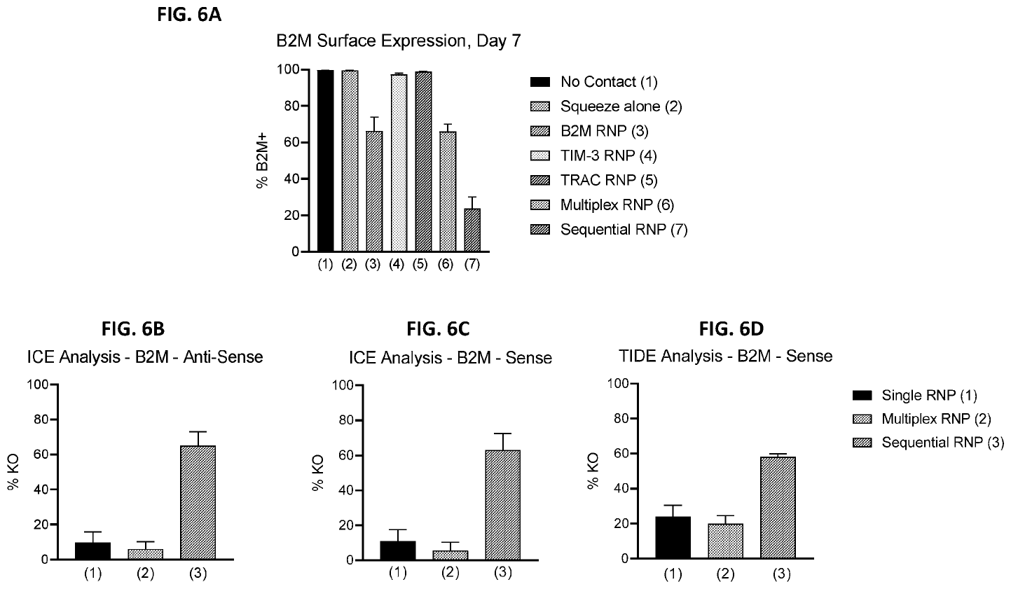

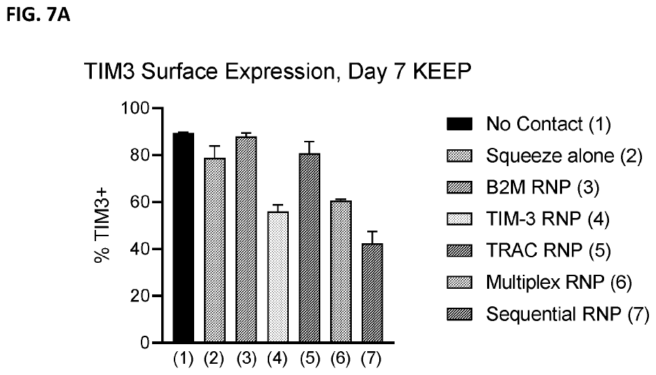

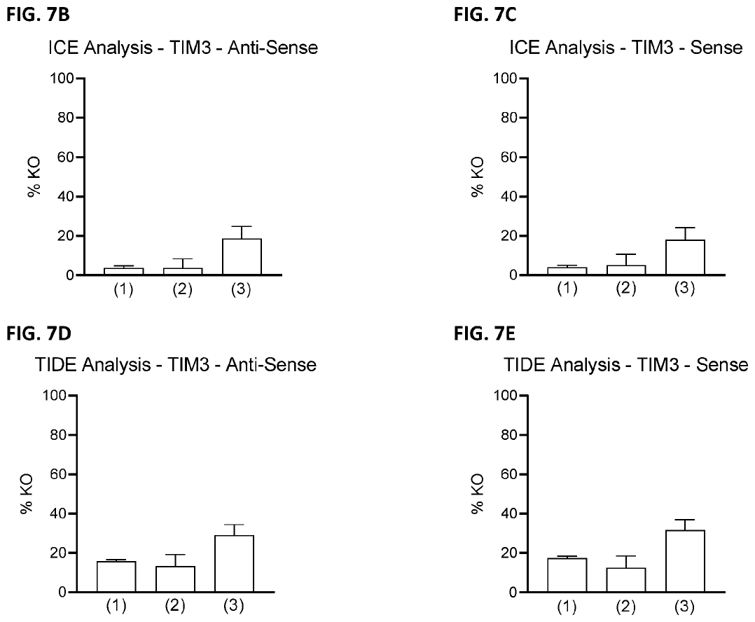

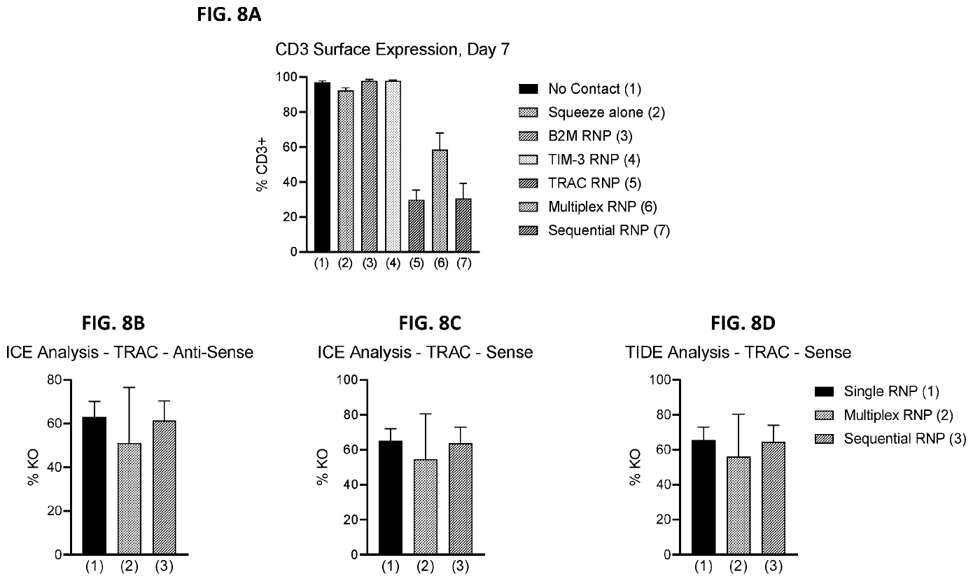

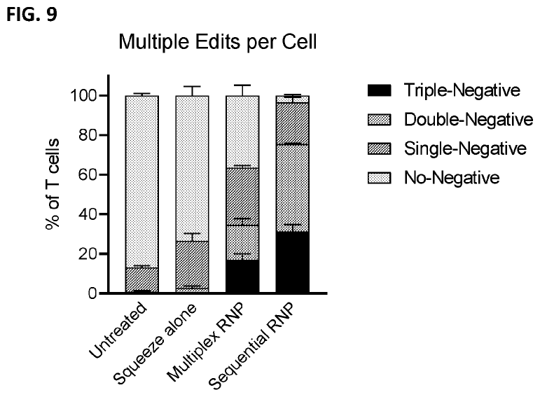

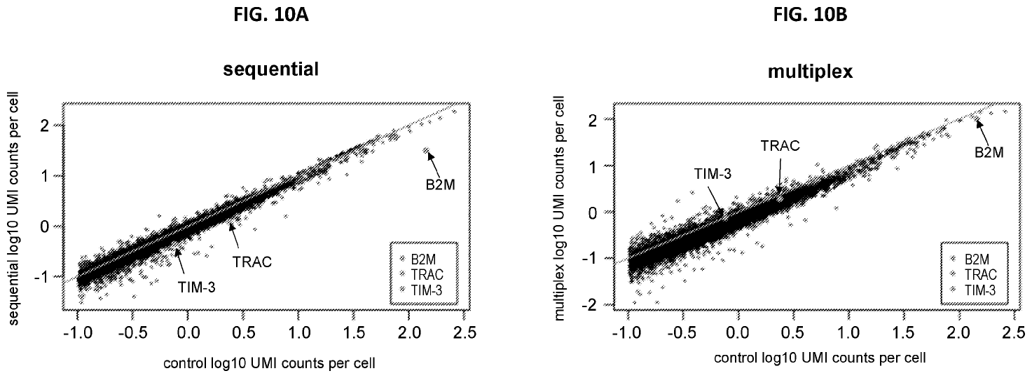

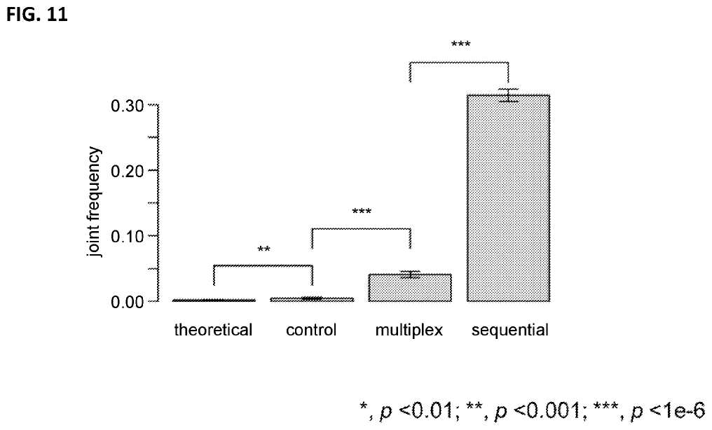

Equivalent or better editing efficiencies were observed in sequentially edited samples compared to single RNP and multiplexed (ceo-delivered) RNP samples. Surface staining for B2M and TIM3 demonstrates more efficient editing in sequential samples, which is confirmed by sequencing analysis (FIGS. 6A-6D and 7A-7E). Note that in this experiment TIM3 surface staining was higher basally than in previous experiments, which allowed for an easier surface readout, particularly when checking for efficiency of attaining multiple edits per cell. CD3 surface staining was used as a proxy for TRAC surface knock down, and demonstrates equivalent surface levels to single RNP editing although multiplexed samples display poorer knockdown (FIG. 8A). Sequencing analysis suggests equivalent TRAC knock out efficiencies across all samples (FIGS. 8B-8D). Surface staining was analyzed using Boolean gating to identify multi-negative populations. Multiplexed, or co-delivered, RNP samples averaged 15% triple negative, whereas sequentially edited samples averaged 35% triple negative (FIG. 9). It is important to note here that untreated and squeeze alone samples have only 15-30% single negative, indicating that surface staining levels were high for all three markers at a basal level, and giving a high degree of confidence in triple negative cells being multi-edited. Better editing efficiency using the sequential method compared to multiplexed was confirmed by deep sequencing using the 5′ 10× Genomics kit, as an analysis of the gene expression levels in comparison to control show that target gene expression in sequentially edited samples deviates farther from control than multiplex edited samples (FIGS. 10A and 10B). Triple knock out efficiency at the genomic level is significantly greater than triple negative events in the control sample using both methods, and sequential delivery results in significantly greater triple knock outs than co-delivery as shown by calculating the number of cells with expression below the threshold for each of the target genes (FIG. 11). These data suggest that while both co-delivery and sequential delivery result in the desired multi-edited phenotype, multi editing through the sequential squeeze method over a course of four days is more effective at achieving larger populations of multi edited cells than co-delivery of all three RNPs.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific aspects have been illustrated and described, the above specification is not restrictive. It will be appreciated that various changes can be made without departing from the spirit and scope of the disclosure(s). Many variations will become apparent to those skilled in the art upon review of this specification.

What is claimed is:

1. A method of delivering a payload to the nucleus of a cell, comprising passing a cell suspension comprising (i) the cell and (ii) the payload through a plurality of constrictions under one or more parameters, wherein passing the cell suspension through the plurality of constrictions allows the payload to enter the cell and be delivered to the nucleus.

2. The method of claim 1, wherein the payload comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

3. The method of claim 1 or 2, wherein the payload comprises a protein-nucleic acid complex.

4. The method of any one of claims 1 to 3, wherein the payload comprises a gene editing tool.

5. The method of claim 4, wherein the gene editing tool comprises a shRNA, siRNA, miRNA, antisense oligonucleotides, zinc-finger nuclease, meganuclease, transcription activator-like effector nuclease (TALEN), a CRISPR/Cas system, a ribonucleoprotein (RNP), a Cre recombinase, a lipid nanoparticle, or any combination thereof.

6. The method of claim 5, wherein the gene editing tool is a CRISPR/Cas system.

7. TCRISPR/Cas system comprises a Cas9 nuclease.

8. The method of any one of claims 3 to 7, wherein the protein-nucleic complex comprises a ribonucleotide protein and a mRNA.

9. The method of any one of claims 4 to 8, wherein the gene editing tool is capable of modulating the expression of a gene selected from a beta-2 microglobulin (B2M), a T-cell immunoglobulin and mucin-domain containing-3 (TIM3), a T-cell receptor alpha constant (TRAC), CD86, TGF-β, PD-1, BC11a, CCR5, CD38, CISH, or combinations thereof.

10. The method of any one of claims 1 to 9, wherein the delivery of the payload to the nucleus occurs less than about 1 hour, less than about 50 minutes, less than about 40 minutes, less than about 30 minutes, less than about 20 minutes, less than about 10 minutes, less than about 5 minutes, less than about 4 minutes, less than about 3 minutes, less than about 2 minutes, less than about 1 minute, less than about 30 seconds, less than about 20 seconds, less than about 10 seconds, less than about 5 seconds, or less than about 1 second after the payload enters the cell.

11. A method of increasing the delivery efficiency of a payload to the nucleus of a cell, comprising passing a cell suspension comprising (i) the cell and (ii) the payload through a plurality of constrictions under one or more parameters, wherein passing the cell suspension through the plurality of constrictions allows greater delivery of the payload to the nucleus of the cell.

12. The method of claim 3, wherein the delivery efficiency of the payload to the nucleus of the cell is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, compared to a reference delivery efficiency.

13. The method of claim 4, wherein the reference delivery efficiency comprises: (i) the delivery efficiency of the payload to the nucleus of the cell after passing the cell through a single constriction; (ii) the delivery efficiency of the payload when delivered to the nucleus using a method that does not comprising passing the cell through a constriction; or (iii) both (i) and (ii).

14. The method of any one of claims 11 to 13, wherein the payload comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

15. The method of any one of claims 11 to 14, wherein the payload comprises a protein-nucleic acid complex.

16. The method of any one of claims 11 to 15, wherein the payload comprises a gene editing tool.

17. The method of any one of claims 11 to 16, wherein the gene editing tool comprises a shRNA, siRNA, miRNA, antisense oligonucleotides, zinc-finger nuclease, meganuclease, transcription activator-like effector nuclease (TALEN), a CRISPR/Cas system, a ribonucleoprotein (RNP), a Cre recombinase, a lipid nanoparticle, or any combination thereof.

18. The method of claim 17, wherein the gene editing tool is a CRISPR/Cas system.

19. TCRISPR/Cas system comprises a Cas9 nuclease.

20. The method of any one of claims 15 to 19, wherein the protein-nucleic complex comprises a ribonucleotide protein and a mRNA.

21. The method of any one of claims 16 to 20, wherein the gene editing tool is capable of modulating the expression of a gene selected from a beta-2 microglobulin (B2M), a T-cell immunoglobulin and mucin-domain containing-3 (TIM3), a T-cell receptor alpha constant (TRAC), or combinations thereof.

22. The method of any one of claims 1 to 21, wherein the plurality of constrictions are contained within a single microfluidic chip.

23. The method of any one of claims 1 to 21, wherein the plurality of constrictions are contained within multiple microfluidic chips, wherein each of the multiple microfluidic chips comprises a constriction.

24. The method of claim 23, wherein each of the multiple microfluidic chips are the same.

25. The method of claim 23, wherein one or more of the multiple microfluidic chips are different.

26. The method of any one of claims 1 to 25, wherein the time interval between passing the cell suspension through a first constriction and a second constriction of the plurality of constrictions is less than about 1 μs, less than about 1 second, less than about 1 minute, less than about 30 minutes, less than about 1 hour, less than about 6 hours, less than about 12 hours, less than about 1 day, less than about 2 days, less than about 3 days, less than about 4 days, or less than about 5 days.

27. The method of any one of claims 1 to 26, wherein the plurality of constrictions comprises at least about 2, at least about 3, at least about 4, at least about 5, at least about 6, at least about 7, at least about 8, at least about 9, at least about 10, at least about 20, at least about 30, at least about 40, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1,000 or more separate constrictions.

28. The method of claim 27, wherein each of the plurality of constrictions is the same.

29. The method of claim 27, wherein one or more of the plurality of constrictions are different.

30. The method of claim 29, wherein the one or more of the plurality of constrictions differ in their length, depth, width, or combinations thereof.

31. A method of concurrently delivering multiple payloads to a cell, comprising passing a cell suspension comprising (i) the cell and (ii) the multiple payloads through a constriction under one or more parameters, wherein passing the cell suspension through the constriction allows the multiple payloads to enter the cells.

32. The method of claim 31, wherein the multiple payloads comprise about 2, about 3, about 4, about 5, about 6, about 7, about 8, about 9, or about 10 types of payloads.

33. The method of claim 31 or 32, wherein each of the multiple payloads is different.

34. The method of any one of claims 31 to 33, wherein the multiple payloads comprise a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

35. The method of any one of claims 31 to 34, wherein the multiple payloads comprise a protein-nucleic acid complex.

36. The method of any one of claims 31 to 35, wherein the payload comprises a gene editing tool.

37. The method of claim 36, wherein the gene editing tool comprises a shRNA, siRNA, miRNA, antisense oligonucleotides, zinc-finger nuclease, meganuclease, transcription activator-like effector nuclease (TALEN), a CRISPR/Cas system, a ribonucleoprotein (RNP), a Cre recombinase, a lipid nanoparticle, or any combination thereof.

38. The method of claim 37, wherein the gene editing tool is a CRISPR/Cas system.

39. TCRISPR/Cas system comprises a Cas9 nuclease.

40. The method of any one of claims 35 to 39, wherein the protein-nucleic complex comprises a ribonucleotide protein and a mRNA.

41. The method of any one of claims 36 to 40, wherein the gene editing tool is capable of modulating the expression of a gene selected from a beta-2 microglobulin (B2M), a T-cell immunoglobulin and mucin-domain containing-3 (TIM3), a T-cell receptor alpha constant (TRAC), or combinations thereof.

42. A method of sequentially delivering a first payload and a second payload to a cell, comprising passing a cell suspension comprising (i) the cell and (ii) the first and second payloads through a first constriction and a second constriction under one or more parameters,
wherein passing the cell suspension through the first constriction allows the first payload to enter the cell, and
wherein passing the cell suspension through the second constriction allows the second payload to enter the cell.

43. The method of claim 42, wherein the cell suspension is passed through the second constriction at least about 1 minute, at least about 30 minutes, at least about 1 hour, at least about 6 hours, at least about 12 hours, at least about 1 day, at least about 2 days, or at least about 3 days after the cell suspension is passed through the first constriction.

44. The method of claim 42 or 43, wherein the first payload, the second payload, or both the first and second payloads comprise a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

45. The method of any one of claims 42 to 44, wherein the first payload, the second payload, or both the first and second payloads comprise a protein-nucleic acid complex.

46. The method of any one of claims 42 to 45, wherein the first payload, the second payload, or both the first and second payloads comprise a gene editing tool.

47. The method of claim 46, wherein the gene editing tool comprises a shRNA, siRNA, miRNA, antisense oligonucleotides, zinc-finger nuclease, meganuclease, transcription activator-like effector nuclease (TALEN), a CRISPR/Cas system, a ribonucleoprotein (RNP), a Cre recombinase, a lipid nanoparticle, or any combination thereof.

48. The method of claim 47, wherein the gene editing tool is a CRISPR/Cas system.

49. TCRISPR/Cas system comprises a Cas9 nuclease.

50. The method of any one of claims 45 to 49, wherein the protein-nucleic complex comprises a ribonucleotide protein and a mRNA.

51. The method of any one of claims 46 to 50, wherein the gene editing tool is capable of modulating the expression of a gene selected from a beta-2 microglobulin (B2M), a T-cell immunoglobulin and mucin-domain containing-3 (TIM3), a T-cell receptor alpha constant (TRAC), or combinations thereof.

52. The method of any one of claims 46 to 51, wherein the first payload and the second payload are different.

53. The method of any one of claims 46 to 51, wherein the first payload and the second payload are the same.

54. The method of any one of claims 46 to 53, wherein the first constriction and the second constriction are different.

55. The method of any one of claims 46 to 53, wherein the first constriction and the second constriction are the same.

56. The method of any one of claims 46 to 55, wherein the first constriction and the second constriction are contained with a single microfluidic chip.

57. The method of any one of claims 46 to 55, wherein the first constriction and the second constriction are contained within separate microfluidic chips.

58. The method of any one of claims 46 to 57, wherein the cell is not in contact with the second payload when the cell suspension is passed through the first constriction.

59. The method of any one of claims 46 to 58, wherein the cell is not in contact with the first payload when the cell suspension is passed through the second constriction.

60. A method of modulating the expression of a gene in a cell, comprising passing a cell suspension comprising (i) the cell and (ii) a payload through a plurality of constrictions under one or more parameters, wherein passing the cell suspension through the plurality of constrictions allows the payload to enter the cell and be delivered to the nucleus of the cell, and wherein the payload is capable of modulating the expression of the gene.

61. The method of claim 60, wherein the payload comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

62. The method of claim 60 or 61, wherein the payload comprises a protein-nucleic acid complex.

63. The method of any one of claims 60 to 62, wherein the payload comprises a gene editing tool.

64. The method of claim 63, wherein the gene editing tool comprises a shRNA, siRNA, miRNA, antisense oligonucleotides, zinc-finger nuclease, meganuclease, transcription activator-like effector nuclease (TALEN), a CRISPR/Cas system, a ribonucleoprotein (RNP), a Cre recombinase, a lipid nanoparticle, or any combination thereof.

65. The method of claim 64, wherein the gene editing tool is a CRISPR/Cas system.

66. TCRISPR/Cas system comprises a Cas9 nuclease.

67. The method of any one of claims 62 to 66, wherein the protein-nucleic complex comprises a ribonucleotide protein and a mRNA.

68. The method of any one of claims 63 to 67, wherein the gene editing tool is capable of modulating the expression of a gene selected from a beta-2 microglobulin (B2M), a T-cell immunoglobulin and mucin-domain containing-3 (TIM3), a T-cell receptor alpha constant (TRAC), or combinations thereof.

69. The method of any one of claims 63 to 68, wherein the expression of the gene in the cell is reduced by at least about 5%, at least about 10%, at least about 20%, at least about 30%, at least about 40%, at least about 50%, at least about 60%, at least about 70%, at least about 80%, at least about 90% or more after the delivery of the payload to the nucleus of the cell.

70. The method of any one of claims 63 to 68, wherein the expression of the gene in the cell is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold or more after the delivery of the payload to the nucleus of the cell.

71. The method of any one of claims 63 to 68, wherein the time interval between passing the cell suspension through a first constriction and a second constriction of the plurality of constrictions is at least about 1 minute, at least about 30 minutes, at least about 1 hour, at least about 6 hours, at least about 12 hours, at least about 1 day, at least about 2 days, or at least about 3 days.

72. The method of any one of claims 63 to 71, wherein the plurality of constrictions comprises at least about 2, at least about 3, at least about 4, at least about 5, at least about 6, at least about 7, at least about 8, at least about 9, at least about 10, at least about 20, at least about 30, at least about 40, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1,000 or more separate constrictions.

73. The method of claim 72, wherein each of the plurality of constrictions is the same.

74. The method of claim 72, wherein one or more of the plurality of constrictions are different.

75. The method of claim 74, wherein the one or more of the plurality of constrictions differ in their length, depth, width, or combinations thereof.

76. The method of any one of claims 1 to 75, wherein the cell comprises a stem cell, a somatic cell, or both.

77. The method of claim 76, wherein the stem cell comprises an induced pluripotent stem cell (iPSC), an embryonic stem cell, a tissue-specific stem cell, a mesenchymal stem cell, or combinations thereof.

78. The method of claim 76, wherein the somatic cell comprises a blood cell.

79. The method of claim 78, wherein the blood cell comprises PBMC.

80. The method of claim 79, wherein the PBMC comprises an immune cell.

81. The method of claim 80, wherein the immune cell comprises a T cell, a B cell, a natural killer (NK) cell, a dendritic cell (DC), a NKT cell, a mast cell, a monocyte, a macrophage, a basophil, an eosinophil, a neutrophil, a DC2.4 dendritic cell, or combinations thereof.

82. The method of any one of claims 1 to 81, wherein the one or more parameters are selected from a cell density; pressure; length, width, and/or depth of the constriction; diameter of the constriction; diameter of the cells; temperature; entrance angle of the constriction; exit angle of the constriction; length, width, and/or width of an approach region; surface property of the constriction (e.g., roughness, chemical modification, hydrophilic, hydrophobic); operating flow speed; payload concentration; viscosity, osmolarity, salt concentration, serum content, and/or pH of the cell suspension; time in the constriction; shear rate in the constriction; type of payload, or combinations thereof.

83. The method of claim 82, wherein the cell density is at least about 1×10³ cells/mL, at least about 1×10⁴ cells/mL, at least about 1×10⁵ cells/mL, at least about 1×10⁶ cells/mL, at least about 2×10⁶ cells/mL, at least about 3×10⁶ cells/mL, at least about 4×10⁶ cells/mL, at least about 5×10⁶ cells/mL, at least about 6×10⁶ cells/mL, at least about 7×10⁶ cells/mL, at least about 8×10⁶ cells/mL, at least about 9×10⁶ cells/mL, at least about 6×10⁷ cells/mL, at least about 7×10⁷ cells/mL, at least about 8×10⁷ cells/mL, at least about 9×10⁷ cells/mL, at least about 1×10⁸ cells/mL, at least about 1.1×10⁸ cells/mL, at least about 1.2×10⁸ cells/mL, at least about 1.3×10⁸ cells/mL, at least about 1.4×10⁸ cells/mL, at least about 1.5×10⁸ cells/mL, at least about 2.0×10⁸ cells/mL, at least about 3.0×10⁸ cells/mL, at least about 4.0×10⁸ cells/mL, at least about 5.0×10⁸ cells/mL, at least about 6.0×10⁸ cells/mL, at least about 7.0×10⁸ cells/mL, at least about 8.0×10⁸ cells/mL, at least about 9.0×10⁸ cells/mL, or at least about 1.0×10⁹ cells/mL or more.

84. The method of claim 82 or 83, wherein the pressure is at least about 1 psi, at least about 2 psi, at least about 3 psi, at least about 4 psi, at least about 5 psi, at least about 6 psi, at least about 7 psi, at least about 8 psi, at least about 9 psi, at least about 10 psi, at least about 20 psi, at least about 30 psi, at least about 35 psi, at least about 40 psi, at least about 45 psi, at least about 50 psi, at least about 55 psi, at least about 60 psi, at least about 65 psi, at least about 70 psi, at least about 75 psi, at least about 80 psi, at least about 85 psi, at least about 90 psi, at least about 95 psi, at least about 100 psi, at least about 110 psi, at least about 120 psi, at least about 130 psi, at least about 140 psi, or at least about 150 psi.

85. The method of any one of claims 82 to 84, wherein the diameter of the constriction is about 20%, about 30%, about 40%, about 50%, about 60%, about 70%, about 80%, about 90%, or about 99% of the diameter of the cell.

86. The method of any one of claims 82 to 85, wherein the length of the constriction is up to 100 μm.

87. The method of claim 86, wherein the length of the constriction is less than about 1 μm, less than about 5 μm, less than about 10 μm, less than about 20 μm, less than about 30 μm, less than about 40 μm, less than about 50 μm, less than about 60 μm, less than about 70 μm, less than about 80 μm, less than about 90 μm, or less than about 100 μm.

88. The method of claim 86, wherein the length of the constriction is about 1 μm, about 5 μm, about 10 μm, about 20 μm, about 30 μm, about 40 μm, about 50 μm, about 60 μm, about 70 μm, about 80 μm, about 90 μm, or about 100 μm.

89. The method of any one of claims 82 to 88, wherein the width of the constriction is up to about 10 μm.

90. The method of claim 89, wherein the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm.

91. The method of claim 89, wherein the width of the constriction is between about 2 μm to about 10 μm.

92. The method of claim 89, wherein the width of the constriction is about 2 μm, about 3 μm, about 4 μm, about 5 μm, about 6 μm, about 7 μm, about 8 μm, about 9 μm, or about 10 μm.

93. The method of any one of claims 82 to 92, wherein the depth of the constriction is at least about 1 μm.

94. The method of claim 93, wherein the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm.

95. The method of claim 93, wherein the depth of the constriction is about 5 μm to about 90 μm.

96. The method of claim 93, wherein the depth is about 5 μm, about 10 μm, about 20 μm, about 30 μm, about 40 μm, about 50 μm, about 60 μm, about 70 μm, about 80 μm, or about 90 μm.

97. The method of any one of claims 82 to 96, wherein the length of the constriction is about 30 μm, the width of the constriction is about 4 μm, and the depth of the constriction is about 70 μm.

98. A cell comprising one or more payloads, wherein the one or more payloads were delivered to the cell using the method of any one of claims 1 to 97.

99. A composition comprising the cell of claim 98, and a pharmaceutically acceptable carrier.

100. A kit comprising the cell of claim 98, and instructions for use.

101. A method of treating a disease or disorder in a subject in need thereof, comprising administering to the subject the cell of claim 98 or the composition of claim 99.

102. The method of claim 101, wherein the disease or disorder comprises a cancer.

103. A composition comprising a population of cells, which have been modified to comprise one or more payloads, wherein the one or more payloads were delivered to the population of cells using the methods of any one of claims 1 to 97.
